(19) United States
(12) Patent Application Publication
(10) Pub. No.: US 2026/0204339 A1
(43) Pub. Date: Jul. 16, 2026

(54) APPARATUS AND METHODS FOR TARGET-CONDITIONED APTAMER DISCOVERY AND BINDING PREDICTION

(71) Applicants: Sawan Patel, Boston, MA (US); Xiaohu Yao, Cary, NC (US); Adam David Friedman, Durham, NC (US)

(72) Inventors: Sawan Patel, Boston, MA (US); Xiaohu Yao, Cary, NC (US); Adam David Friedman, Durham, NC (US)

(21) Appl. No.: 19/398,384

(22) Filed: Nov. 24, 2025

Related U.S. Application Data
(63) Continuation of application No. 19/272,758, filed on Jul. 17, 2025, now abandoned.
(60) Provisional application No. 63/724,796, filed on Nov. 25, 2024.

(51) Int. Cl. G16B 5/00; G06N 3/08; G16B 30/00; G16B 40/20; G16B 45/00

(52) U.S. Cl. CPC G16B 5/00; G06N 3/08; G16B 30/00; G16B 40/20; G16B 45/00

(57) ABSTRACT

The present disclosure provides computer-implemented methods and apparatus for predicting aptamer-protein binding and for generating target-specific aptamer candidates using a deep learning architecture that integrates transformer-based molecular encoders with self-attention and cross-attention networks. Aptamer and protein sequences are tokenized, vectorized, and embedded using specialized encoders, after which a self-attention network models intramolecular relationships and a cross-attention network models intermolecular interactions. A multi-layer perceptron produces a binding prediction score, and certain embodiments identify nucleotides and residues that contribute most to binding. The apparatus may store critical binding motifs, iteratively optimize aptamer sequences, and visualize predicted interfaces. Training methods employ curated datasets with experimentally measured affinities, gradient-based optimization, and validation on held-out sets. A generative model, using Monte Carlo search, diffusion, or other conditional generation methods, produces candidate aptamers that are rapidly screened by the classifier. This integrated framework enables high-throughput, accurate, and computationally efficient discovery of target-specific aptamers.

### CROSS-REFERENCE DATA

This patent application claims a priority date benefit from the co-pending U.S. Provisional Patent Application No. 63/724,796 filed on Nov. 25, 2024, and entitled APTAMER DISCOVERY USING A LARGE LANGUAGE MODEL CLASSIFIER. This patent application is also a continuation-in-part of the co-pending U.S. patent application Ser. No. 19/272,758, filed on Jul. 17, 2025, entitled TUNABLE DENSITY OF CELL-TARGETING LIGANDS ON LIPID NANOPARTICLES FOR TARGETED DELIVERY. Both documents are incorporated herein by reference in their respective entireties.

### SEQUENCE LISTING XML

A Sequence Listing XML file entitled “aptable-seq-final.xml” has been created on Nov. 24, 2025, and submitted electronically via the USPTO's EFS-Web system on the same date. The size of the file is 17,034 Bytes. The content of the Sequence Listing is incorporated herein by reference in its entirety. The Sequence Listing contains the nucleotide sequences necessary to understand the invention and is provided in both the application as filed and in the accompanying XML file.

### BACKGROUND

Without limiting the scope of the invention, its background is described in connection with aptamers. More particularly, the invention describes improvements to an apparatus and methods concerning the design of an aptamer, oligo-based binder that can bind specifically to a protein target, including DNA and RNA aptamers. Improved quality and speed of the aptamer discovery would be beneficial for rapid disease diagnostics and improved therapeutic outcomes.

Aptamers are artificial oligonucleotides that can bind specifically to target molecules, offering advantages over traditional antibodies, including lower production costs, better stability, and easier modification. Historically, aptamers have been discovered through SELEX (Systematic Evolution of Ligands by Exponential Enrichment), an experimental process that is time-consuming and resource-intensive. The growing importance of aptamers in diagnostics, therapeutics, and biosensing has created an urgent need for computational methods to accelerate their development. Traditional computational approaches have focused on structure prediction and simple sequence-based methods, but these have limited accuracy in predicting binding interactions.

A challenge in aptamer development is accurately predicting binding interactions between aptamers and their protein targets. Traditional methods struggle to capture complex molecular properties that determine binding success, including charge distribution, structural stability, and interaction interfaces. Additionally, existing computational approaches often require extensive experimental validation and are limited in their ability to generalize across different types of aptamer-protein pairs. The field needs methods that can rapidly screen large numbers of candidates while maintaining high prediction accuracy. Additionally, aptamer discovery could be accelerated by and, possibly, fully reliant on in-silico methods for aptamer generation through the use of generative computer modeling. These high-throughput, low-latency methods have the promise of being able to generate whole libraries of nascent aptamers designed to bind to a target.

### SUMMARY

Accordingly, it is an object of the present invention to overcome these and other drawbacks of the prior art by providing a novel apparatus and methods for predicting aptamer-protein target binding.

It is another object of the present invention to provide a target-conditioned candidate aptamer prediction apparatus.

It is a further object of the present invention to provide a method of training a machine learning model for predicting an aptamer-protein target binding.

It is yet a further object of the present invention to provide a computer-implemented generation method for the in-silico discovery of at least one target-specific aptamer.

The present disclosure relates to computer-implemented methods and apparatus for predicting binding interactions between an aptamer and a molecular target protein, as well as for generating target-conditioned aptamer candidates. Embodiments leverage a novel deep learning architecture that integrates specialized molecular encoders with multi-layer self-attention and cross-attention networks to capture complex biochemical properties and interaction patterns directly from sequence data.

In various embodiments, an aptamer sequence and a molecular target protein sequence are tokenized into discrete units and converted into corresponding numerical vectors. These vectors are processed by an aptamer encoder and a molecular target protein encoder. Each encoder may be optionally implemented as a transformer-based model tailored for biological sequences, to generate high-dimensional embeddings representing structural, chemical, and contextual features of the respective sequences. A self-attention network models intramolecular relationships within each embedding, while a cross-attention network models intermolecular interactions by relating protein query token embeddings to aptamer key-value embeddings. The outputs of the cross-attention network are optionally processed through a multi-layer perceptron to yield a prediction score indicating the likelihood of binding between the aptamer and the target protein. Certain embodiments further identify nucleotides and amino acid residues contributing most to the predicted interaction, enabling interpretable binding interface visualization.

An apparatus for implementing these methods comprises a processor and a memory storing a machine learning model configured to perform vectorization, encoding, self-attention processing, cross-attention processing, and binding score prediction. The memory may further store representations of critical motifs relevant to protein target-binding, while the processor may iteratively modify aptamer sequences to optimize predicted binding scores. An input/output module may be provided to receive sequence inputs and present visualizations of predicted binding interactions.

Further embodiments describe methods of training the machine learning model using curated datasets comprising known aptamer-protein target pairs, including negative examples and sequences with experimentally measured binding affinities. Training includes generating vector representations and embeddings, processing them through the attention networks, comparing predicted scores to labeled binding data, and updating model parameters via gradient-based optimization and backpropagation. Model performance may additionally be validated on held-out test sets and fine-tuned for particular target protein families or aptamer classes.

The disclosure also provides computer-implemented methods for in-silico discovery of target-specific aptamers. A generative model may be implemented via Monte Carlo tree search, Monte Carlo tree guidance, random sequence generation, or a masked discrete diffusion model. The generative model may be conditioned on a target protein (and optionally one or more non-target proteins) to produce candidate aptamer sequences. These candidates are rapidly evaluated using the cross-attention-based classifier, enabling high-throughput screening of large libraries. By integrating generative modeling with a deep learning classifier, the system generates aptamer sequences that are not only novel but also predicted to bind the intended molecular target with high selectivity. This computational framework substantially reduces the time, cost, and experimental iterations typically required for aptamer development, while maintaining high predictive accuracy through its sophisticated neural architecture.

In embodiments, a computer-implemented generation method for the in-silico discovery of at least one target-specific aptamer, the method comprising the following steps:
a. receiving a target molecular target protein target sequence,
b. receiving at least one molecular non-target protein sequence,
c. generating a candidate aptamer sequence using a generative model, wherein the generative model is trained to output at least one oligonucleotide sequence having a specified length, and wherein generating the candidate aptamer sequence further comprises a step of using a Monte Carlo tree search algorithm or a Monte Carlo tree guidance algorithm to generate a conditional aptamer sequence predicted to bind the molecular protein target and not bind the at least one non-target molecular protein,
d. determining a binding probability between the generated candidate aptamer sequence and all molecular protein target sequences using a machine learning model comprising a self-attention network and a cross-attention network, and
e. selecting at least one aptamer sequence based on the binding probability of step (d) and predicted kinetics.

The method may also include a further embodiment, wherein step (c) further comprises using a random sequence generation or a masked discrete diffusion model to conditionally generate a protein target-binding aptamer sequence using a step of training a machine learning model for predicting aptamer-protein target binding. The training may include the following steps:
a. curating a training dataset and a benchmark dataset comprising known and proprietary aptamer-protein target pairs and corresponding binding data, in addition to known natural oligonucleotide-target complexes,
b. generating a vector representation of at least one aptamer-protein target pair,
c. generating an aptamer-protein embedding for the at least one aptamer-protein target pair,
d. processing the aptamer-protein embedding through a self-attention network and a cross-attention network,
e. comparing at least one predicted binding score with experimental binding data, and
f. updating at least one parameter of the machine learning model using gradient-based optimization, using the comparison of step (v), thereby minimizing aptamer-protein binding prediction error.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates structural and functional components of a target-conditioned aptamer discovery system, including a vectorization component for converting raw sequence inputs into numerical representations, specialized encoders for generating aptamer and protein embeddings, attention networks for modeling intra- and intermolecular relationships, and a prediction module for producing binding likelihood scores.

FIG. 2 illustrates a process-flow diagram for a target-conditioned aptamer discovery system, showing sequential operations including sequence input, vectorization, embedding generation, neural-network processing, and binding-score computation.

FIG. 3 illustrates an example method for generating vector representations, creating embeddings, and processing aptamer and protein sequences using a target-conditioned aptamer discovery system, including self-attention, cross-attention, and score-generation steps.

FIG. 4 illustrates an example of an aptamer-discovery pipeline that integrates generative methods (including MCTS and masked-diffusion approaches), binding-prediction processing, and validation stages such as molecular-dynamics simulations and wet-lab assays.

FIG. 5 illustrates the generative aspects of the system, including step-wise aptamer generation using MCTS algorithm, secondary-structure prediction of generated aptamers, and predicted docking conformations demonstrating computationally inferred aptamer-protein interactions.

FIG. 6 outlines the MCTS algorithm in pseudocode.

FIG. 7 illustrates the generative aspects of the system, including step-wise aptamer generation using MCTG algorithm, secondary-structure prediction of generated aptamers, and predicted docking conformations demonstrating computationally inferred aptamer-protein interactions.

FIG. 8 outlines the MCTG algorithm in pseudocode.

FIG. 9 demonstrates the invention's integration with analyzing an hCD117-targeted aptamer sequence library.

FIG. 10 demonstrates the invention's integration with analyzing hCD117 aptamer clusters in a traditional bioinformatics workflow.

FIG. 11 demonstrates the invention's blind usage with processing a small subset of aptamers within an amplified sequence library following a hCD117 SELEX procedure

FIG. 12 demonstrates the poor correlation between the invention's predicted binding score with each binding aptamer's Kd when blind to all aptamers and the protein target.

FIG. 13 demonstrates the fine-tuned model's usage with processing a small subset of aptamers within an amplified sequence library following a hCD117 SELEX procedure.

FIG. 14 demonstrates the moderate correlation between the invention's predicted binding score with each binding aptamer's Kd when finetuned with an aptamer known to bind the hCD117 target.

FIG. 15 demonstrates the invention's predicted binding interface between the SARS-CoV-2 spike glycoprotein and aptamer RBD-1C, compared to the true characterized binding interface.

FIG. 16 demonstrates the invention's predicted binding interface between the SARS-CoV-2 spike glycoprotein and aptamer RBD-4C, compared to the true characterized binding interface.

FIG. 17 describes the invention's ability to identify ssDNA binding aptamers from a SELEX library for a protein target, here hCD117, with validated high-affinity.

FIG. 18 describes the invention's ability to generate ssDNA binding aptamers using the MCTS algorithm for targeted lipid nanoparticle delivery.

FIG. 19 describes the invention's ability to de novo generate ssDNA binding aptamers using either the MCTS and MCTG algorithms, for binding a target protein with moderate-to-high affinity.

### DETAILED DESCRIPTION OF PREFERRED EMBODIMENTS OF THE INVENTION

The following description sets forth various examples along with specific details to provide a thorough understanding of the claimed subject matter. It will be understood by those skilled in the art, however, that claimed subject matter may be practiced without one or more of the specific details disclosed herein. Further, in some circumstances, well-known methods, procedures, systems, components, and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

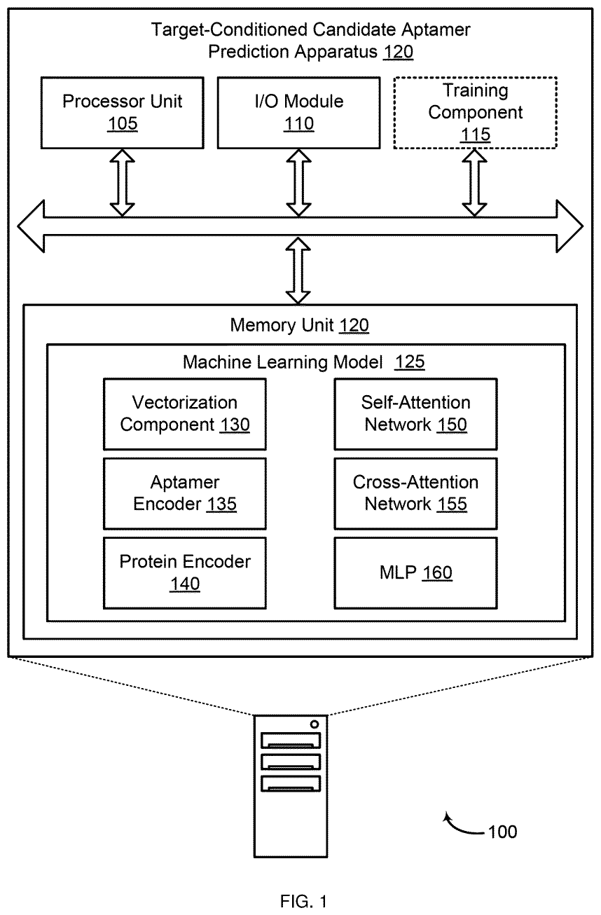

FIG. 1 illustrates an example of Target-Conditioned Candidate Aptamer Prediction Apparatus 120, which generally comprises a memory unit with a machine learning model, a processor unit, an I/O module, and a training component. In this example, the Vectorization Component (130) transforms raw sequence inputs into a machine-readable format. It takes the text strings representing aptamer and protein sequences and converts them into numerical vectors that preserve essential molecular characteristics and chemical properties. This conversion enables subsequent machine learning processing while maintaining biologically relevant information.

The Aptamer Encoder (135) and Protein Encoder (140) process their respective vectorized sequences into high-dimensional embeddings. These specialized encoders capture complex molecular features, including structural patterns, chemical properties, and potential interaction sites. Each encoder is optimized for its specific molecule type while producing compatible embedding formats for downstream processing.

The Self-Attention Network (150) processes embeddings individually to model internal molecular relationships. It analyzes how different parts of each molecule relate to each other, capturing structural dependencies and important molecular features. The self-contextualized representation enhances the model's understanding of each molecule's properties.

The Cross-Attention Network (155) analyzes relationships between the aptamer and protein embeddings to model potential binding interactions. The Cross-Attention Network (155) learns patterns that indicate molecular compatibility and likely binding sites, producing interaction-aware representations that combine information from both molecules.

The Multi-Layer Perceptron (MLP) (160) processes the combined representations from the attention networks to generate a final binding prediction. The Multi-Layer Perceptron (MLP) (160) reduces the complex, high-dimensional features into a single probability score indicating the likelihood of successful binding between the input aptamer and protein sequences. However, embodiments of the present disclosure may not be limited thereto, and structural components other than the Multi-Layer Perceptron (MLP) (160) may be used.

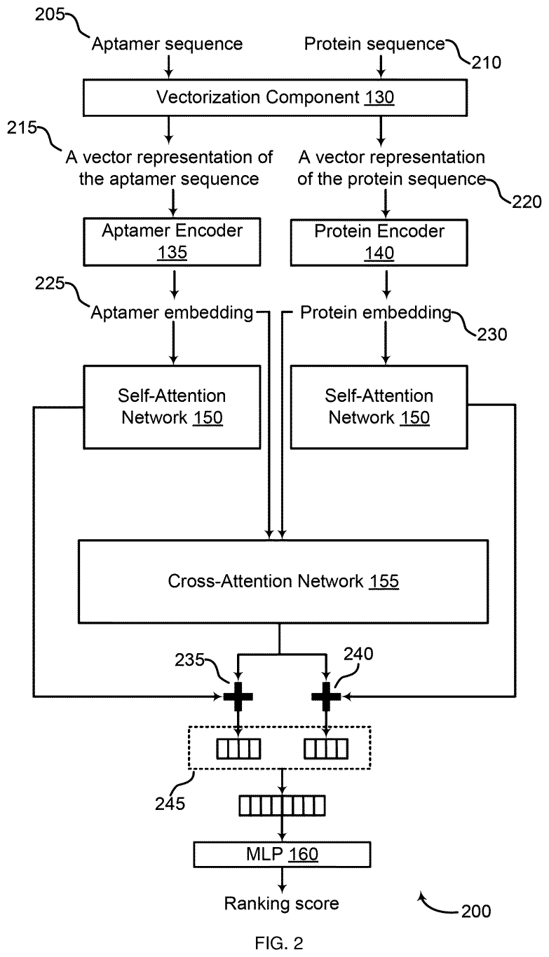

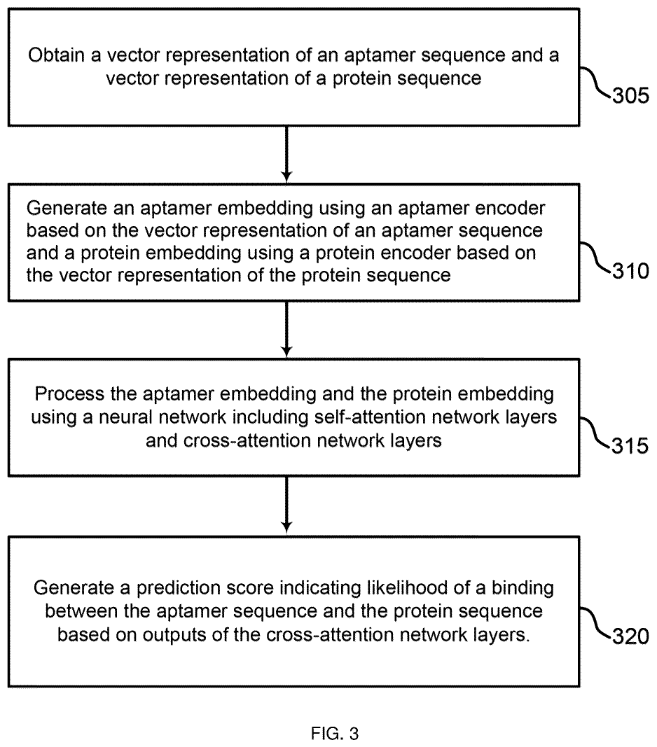

FIG. 2 illustrates an aptamer discovery process according to embodiments of the present disclosure. FIG. 3 illustrates an example of an aptamer discovery method according to embodiments of the present disclosure. At Step 305 (directed to Vector Representation): the system first converts both the aptamer and protein sequences into numerical vector representations suitable for machine learning processing. This vectorization step involves tokenizing the sequences into smaller units and mapping them to corresponding numerical vectors that preserve the chemical and structural properties of the molecules. While specific tokenization methods may be implemented, the invention encompasses various approaches to generating machine-readable vector representations.

At Step 310 (directed to Embedding Generation), the system, by using specialized encoders (such as, but not limited to, RNA-FM/Nucleotide Transformer for aptamers and ESM-2 for proteins), transforms the vector representations into high-dimensional embeddings. These embeddings capture complex molecular properties, including structural information, charge distribution, and stability characteristics essential for predicting binding behavior. The invention contemplates the use of various existing or future encoding technologies capable of generating meaningful molecular embeddings.

At Step 315 (directed to Neural Network Processing): The embeddings are processed through a sophisticated neural network architecture combining self-attention and cross-attention mechanisms. Self-attention layers analyze internal relationships within each molecule's embedding, while cross-attention layers model interactions between the aptamer and protein embeddings, capturing potential binding interfaces and molecular compatibility. The specific architecture and parameters may be modified while maintaining the core concept of dual-attention processing.

At Step 320 (directed to Score Generation): The system generates a final prediction score between 0 and 1 based on the processed embeddings. While one implementation passes the processed embeddings through a multi-layer perceptron (MLP), other architectural choices or processing methods may be employed to generate the binding likelihood score. The score incorporates all the learned molecular interaction patterns and structural compatibility factors analyzed in previous steps, with various possible approaches to final score computation.

Embodiments of the present disclosure enable several important real-world applications in healthcare and biotechnology. In drug discovery, the system can rapidly screen and design therapeutic aptamers targeting specific disease-related proteins. This significantly accelerates the drug development process by identifying promising candidates before expensive experimental validation. The technology also enables the development of personalized medicine approaches by matching aptamer-based drugs to specific protein variants associated with individual patients' conditions.

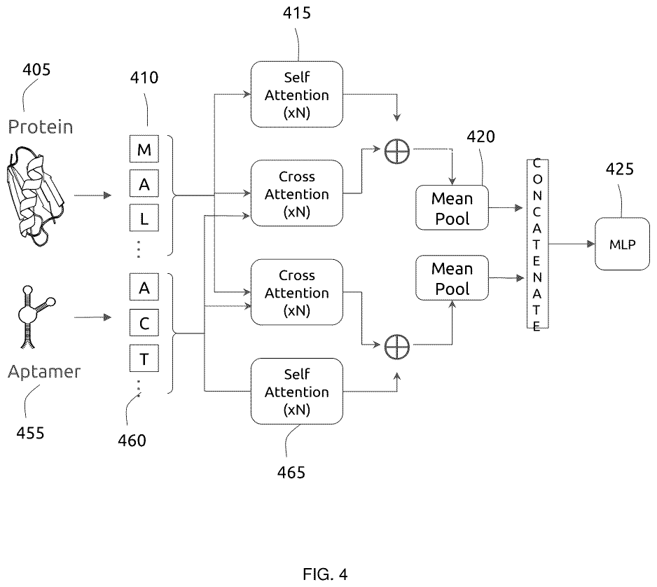

FIG. 4 shows an example of the model architecture. In this example, the flow begins with two sequences—a protein amino acid sequence (405) and an aptamer nucleic acid sequence (455). These two sequences are vectorized (410, 460) and processed through the system for binding prediction and interface analysis. Their respective self-attention and cross-attended products, for the protein (415) and the aptamer (465), are averaged and concatenated (420). Lastly, the result is reduced to a single binding confidence score (425).

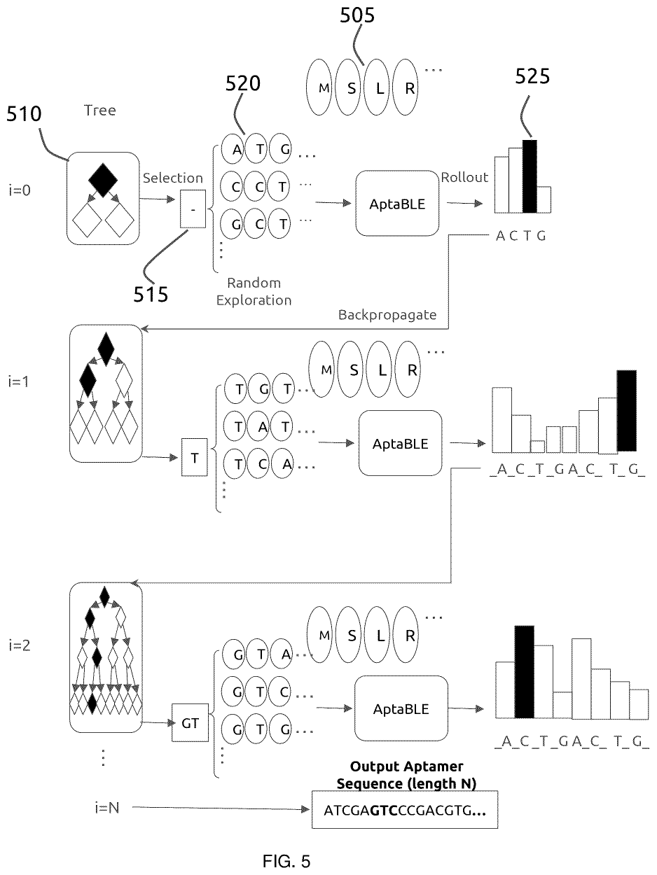

FIG. 5 displays the step-by-step process of aptamer generation using MCTS algorithm, where the system builds sequences sequentially while evaluating binding potential through the system. Initially, a protein amino acid sequence (505) is supplied to the algorithm which initializes an empty tree (510). This tree initially contains an empty root node corresponding with an empty aptamer sequence. This node is selected as the current node (515), from which several child nodes are expanded. From each child node, candidate sequences are randomly constructed according to the desired aptamer length (520). These child nodes, listed in (525), are continually visited and scored using the binding confidence against the target protein. Ultimately, the child node with the highest normalized binding confidence score is selected in the next iteration, after which the procedure repeats from (515).

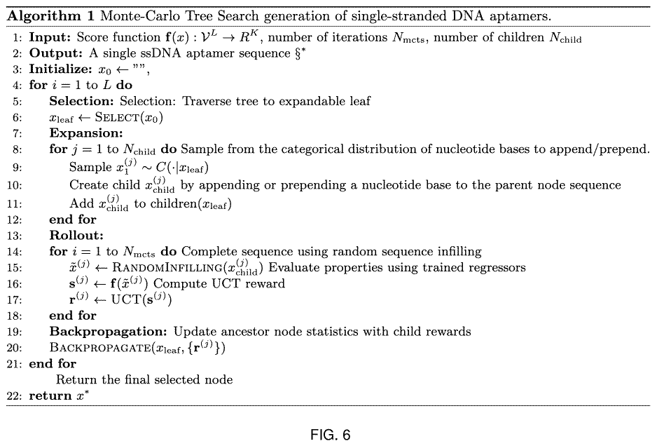

FIG. 6 outlines the MCTS algorithm in pseudocode.

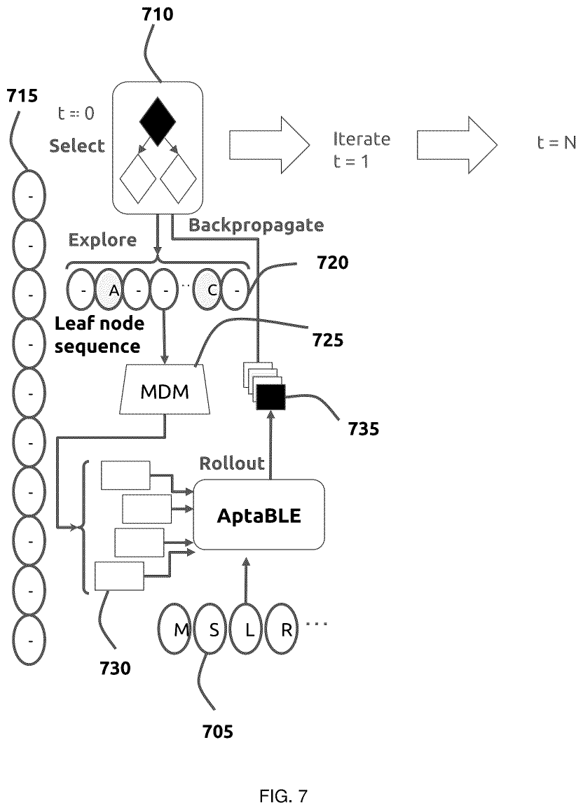

FIG. 7 displays the aptamer generation process using MCTG algorithm, where the system iteratively constructs sequences by progressively uncorrupting a masked sequence to favor sequences with higher binding prediction scores. First, a protein amino acid sequence (705) is supplied to the algorithm which again initializes an empty tree (710). The root node is again an empty aptamer sequence of fixed length, which is selected (715). The root node's child nodes, each corresponding with progressively more filled aptamer sequence, still consistent with the parent root node, are expanded by visitation (720) and sampling from a masked diffusion model (725). These sampled sequences consistent with both the parent root node and the child node, are all scored for target binding confidence (730), which are used to update the child node's normalized score in the tree (735). Ultimately, following a number of iterations, the child node with the highest normalized target binding score is selected as the next parent node, repeating the procedure from (715).

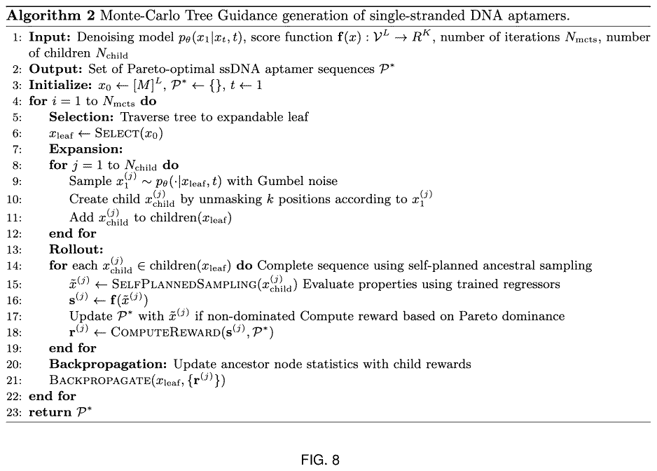

FIG. 8 outlines the MCTG algorithm in pseudocode.

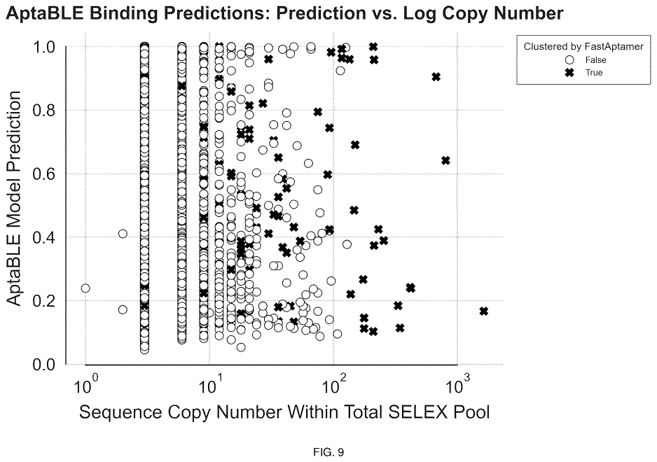

FIG. 9 demonstrates the invention's integration with analyzing a hCD117-targeted aptamer sequence library. Aptamer sequences, in traditional bioinformatics processing practices, are grouped into a predetermined number of clusters where each cluster contains aptamer sequences whose sequential edit distance is no more than 3 from the cluster characteristic sequence. A cluster characteristic sequence is the sequence within the cluster that has the highest number of reads, or frequency, within the experiment's output sequencing data. Here, sequences in white were omitted from a cluster and thereby omitted from the traditional path of analysis, despite their higher predicted binding confidence score as determined. Conversely, black points correspond to sequences that were clustered and further characterized using the traditional bioinformatics workflow. However, several have limited predicted interaction with their intended target in the workflow described in this proposal.

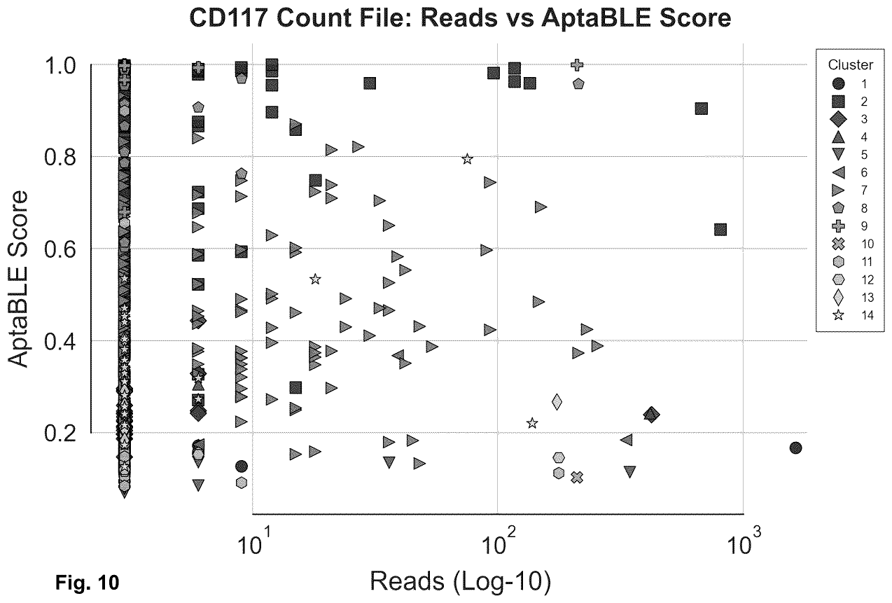

FIG. 10 describes the invention's integration with analyzing hCD117 aptamer clusters in a traditional bioinformatics workflow. Sequences within a cluster, as described in FIG. 9, are highly similar. As such, a single sequence from each cluster is typically synthesized while all other members are ignored in common practice. In this figure, we highlight that leading sequences within each cluster, which are the sequences with the greatest number of reads (furthest to the right per symbol), do not always have the highest predicted binding interaction score. As such, the practice of selecting the most copied sequence within each cluster may not be infallible.

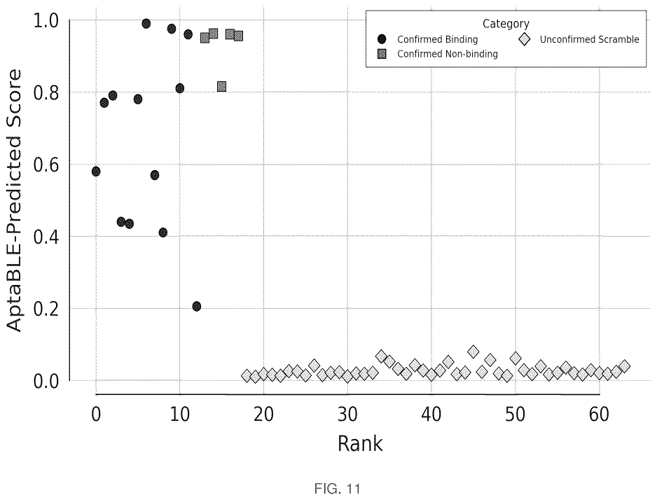

FIG. 11 highlights the invention's blind usage with processing a small subset of aptamers within an amplified sequence library following a hCD117 SELEX. Here, a subset of known binding aptamers to hCD117, represented as circles, are scored with typically high confidence for binding interaction. However, certain aptamers within the SELEX sequence library that are non-binding can also be scored highly, reflected as squares in the figure. These non-binding aptamer-like sequences are in contrast to artificially generated non-binding sequences (diamonds), which are scored very poorly.

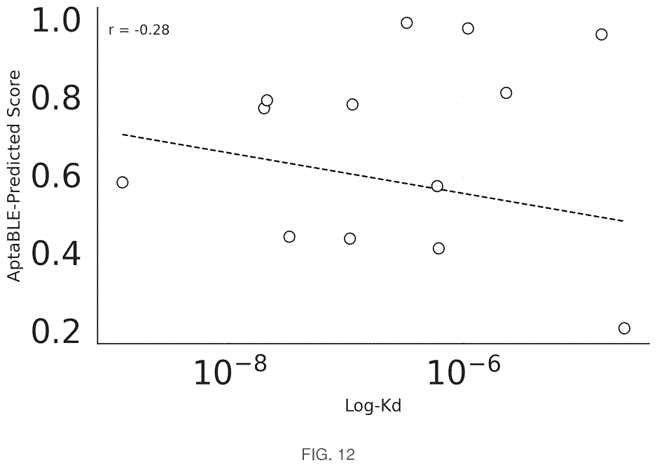

FIG. 12 describes the relatively poor correlation between the invention's predicted binding score with each binding aptamer's Kd when blind to all aptamers and the protein target.

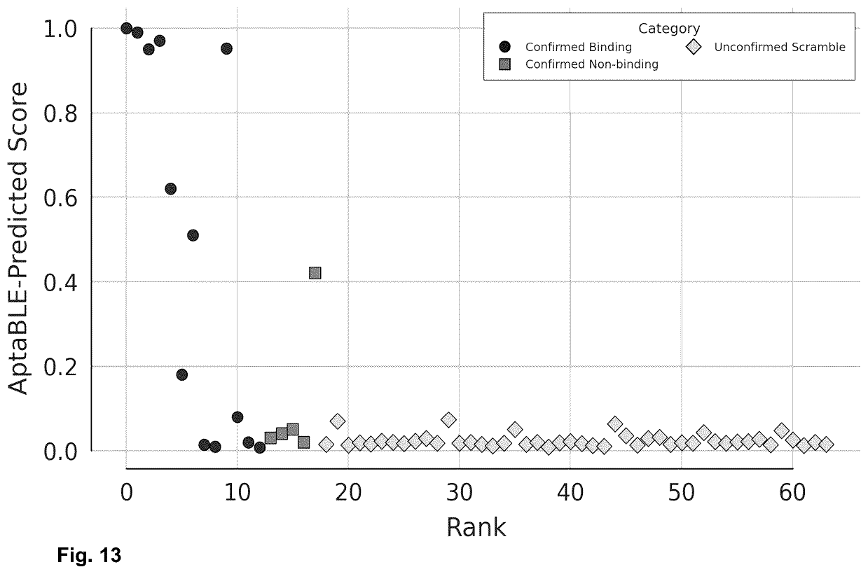

FIG. 13 highlights the fine-tuned invention's usage with processing a small subset of aptamers within an amplified sequence library following a hCD117 SELEX. Following model feedback, aptamers known to bind the hCD117 target (circles) are all scored highly, whereas all negative sequences (squares and diamonds) are scored poorly.

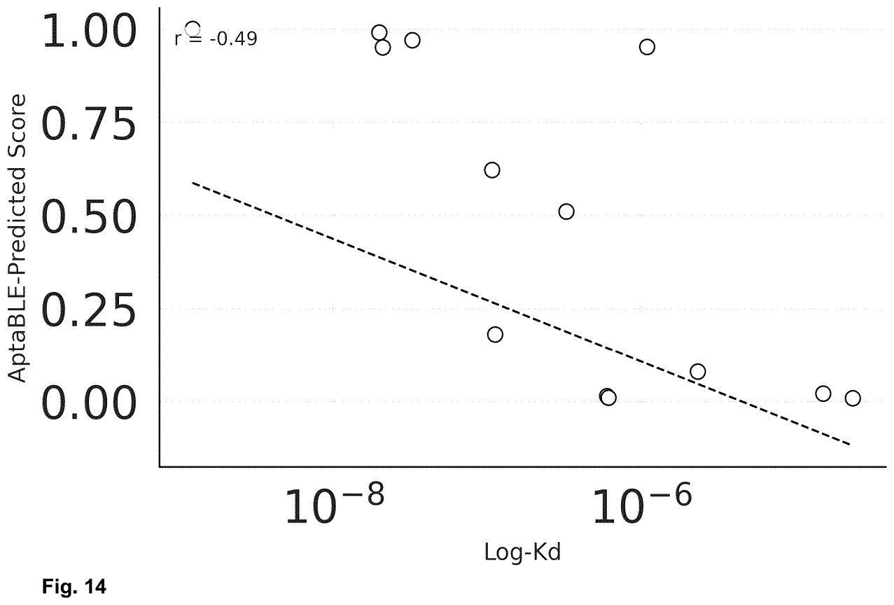

FIG. 14 describes the moderate correlation between the invention's predicted binding score with each binding aptamer's Kd when finetuned with an aptamer known to bind the hCD117 target.

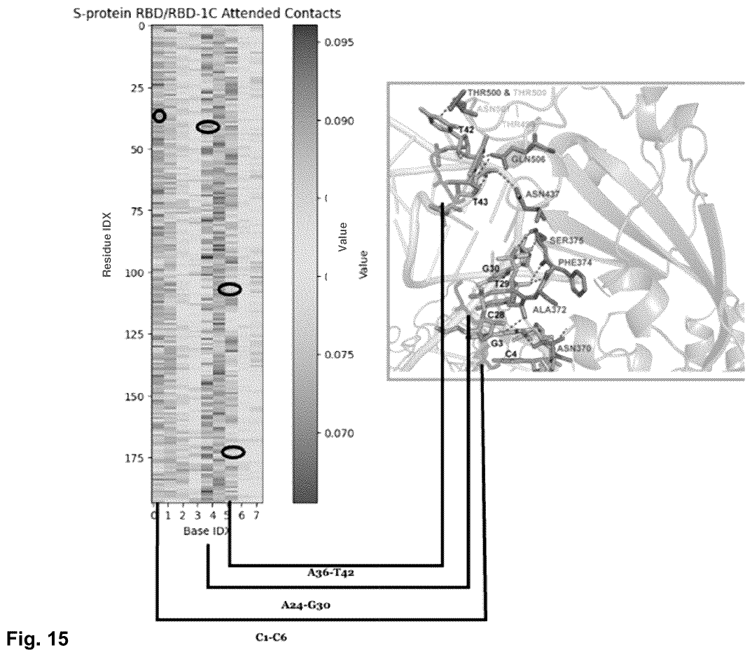

FIG. 15 describes the invention's ability to reproduce a binding interaction map between protein amino acid residues and individual nucleotides in a nucleic acid polymer for a known-binding ssDNA aptamer for SARS-CoV-2 Spike glycoprotein, named RBD-1C.

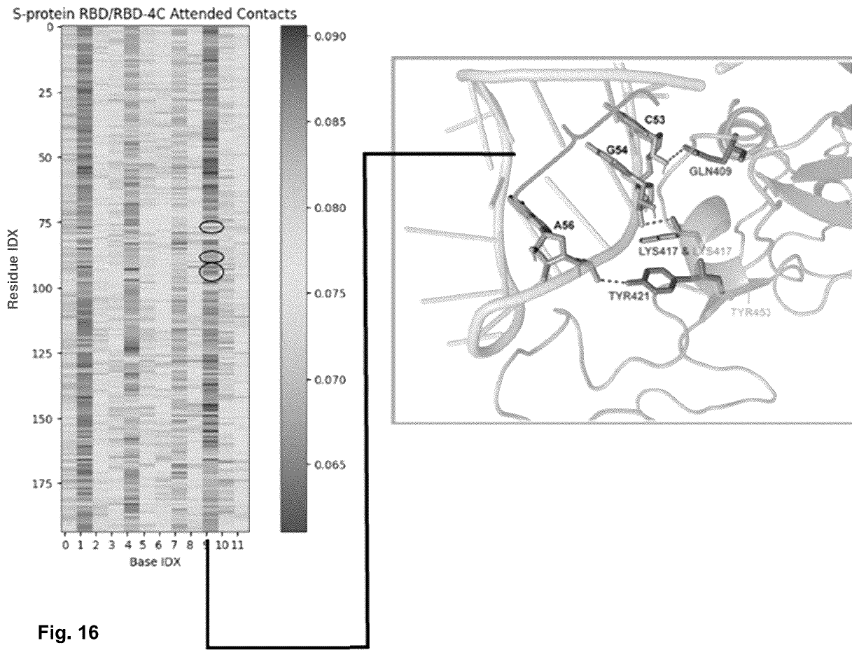

FIG. 16 describes the invention's ability to reproduce a binding interaction map between protein amino acid residues and individual nucleotides in a nucleic acid polymer for a known-binding ssDNA aptamer for SARS-CoV-2 Spike glycoprotein, named RBD-4C.

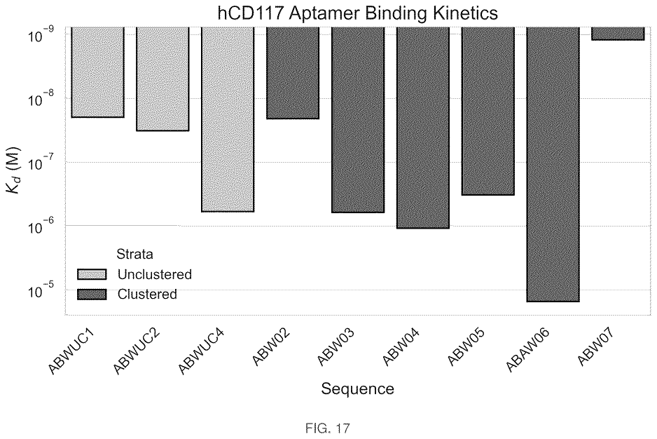

FIG. 17 describes the invention's ability to identify ssDNA binding aptamers from a SELEX library for a protein target, here hCD117, with validated high-affinity. We note ‘high-affinity’ as an aptamer with a sub-50-nanomolar dissociation constant. The aptamers satisfying this criterion are ABWUC1 and ABWUC2.

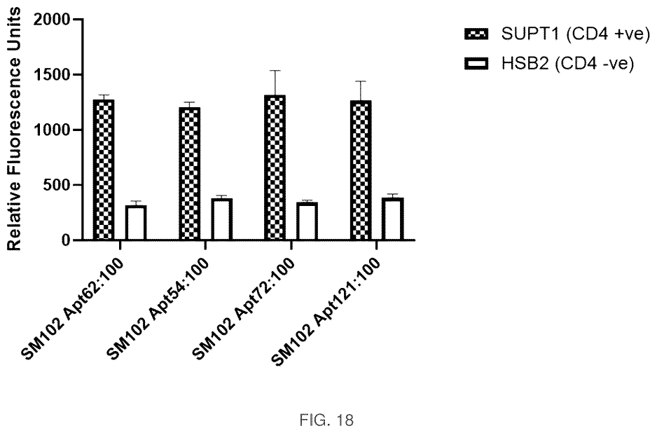

FIG. 18 describes the invention's ability to generate ssDNA binding aptamers using the MCTS algorithm for endowing a lipid nanoparticle with functional targeting specific to a specific human cell surface receptor, here hCD4. Aptamers were conjugated to the surface of a lipid nanoparticle at 1:100 ratios and transfected into both hCD4+SUPT1 cells and hCD4-HSB2 cells. Accordingly, we observe that model-generated sequences Apt. 54, 72, and 121 achieve comparable or superior targeting specificity to the hCD4+ cell type.

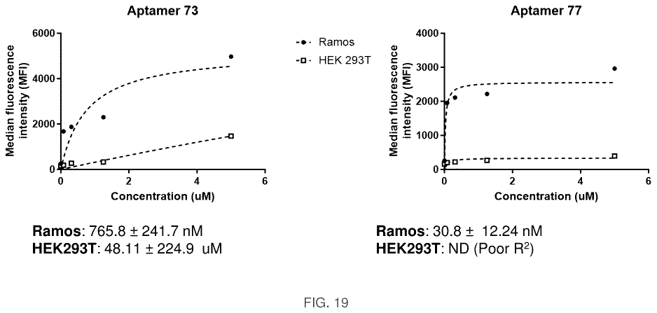

FIG. 19 describes the invention's ability to de novo generate ssDNA binding aptamers using either the MCTS and MCTG algorithms, for binding a target protein with moderate-to-high affinity. We denote ‘moderate-affinity’ as an aptamer with a sub-micromolar dissociation constant and ‘high-affinity’ as an aptamer with a sub-50-nanomolar dissociation constant. The aptamers satisfying these criteria are aptamers 73 and 77, respectively.

In diagnostics, the system can help design highly specific aptamer-based biosensors and diagnostic tools. These could be used for early disease detection by identifying specific protein biomarkers, monitoring disease progression, or screening for various medical conditions. The high-throughput nature of the system allows for rapid development of new diagnostic tests in response to emerging health threats.

The present invention also has valuable applications in research and industrial settings. Research laboratories can use it to study protein-protein interactions and validate potential drug targets. In industrial biotechnology, the system can assist in developing quality control measures, optimizing purification processes, and monitoring manufacturing processes. Environmental applications include developing sensors for detecting toxins, pathogens, or contamination in water, food, and other substances, contributing to public health and safety monitoring efforts.

According to some alternative embodiments, a novel generative pipeline is provided for creating new aptamer sequences with enhanced binding affinity to specific protein targets. The system implements two complementary approaches: a conditional generation method using Monte Carlo tree search (MCTS) and an unconditional generation method using a masked discrete diffusion model (DRLM). The MCTS algorithm directly incorporates target protein information during sequence generation, while the DRLM generates diverse aptamer candidates that are subsequently filtered for target specificity.

In some examples, the MCTS-based approach employs a tree search algorithm that explores the space of possible aptamer sequences while being guided by predicted binding affinities to the target protein. The conditional generation method enables direct optimization of sequences for specific targets, incorporating structural and chemical compatibility factors throughout the generation process. For example, the system can generate both DNA and RNA aptamers, with their secondary structures and predicted docking configurations providing additional validation of binding potential.

In some examples, the DRLM approach provides an alternative generation method using a diffusion model trained on aptamer sequences. This model learns the underlying distribution of valid aptamer sequences and can generate novel candidates through a controlled diffusion process. While these sequences are generated unconditionally, they are subsequently evaluated using the binding prediction model to identify promising candidates for specific targets. The combination of both generation methods provides a robust pipeline for discovering new aptamers with desired binding properties.

The generated sequences from both methods undergo evaluation using the neural network-based binding prediction model, employing attention mechanisms to assess molecular compatibility. This integrated approach combines the advantages of both conditional and unconditional generation strategies, providing a comprehensive system for developing new aptamers for various applications in research, diagnostics, and therapeutics.

In this disclosure, target-conditioned refers to the model's predictions and operations are specifically contextualized by and dependent on the protein target sequence input. The model evaluates aptamer candidates specifically in relation to their potential binding with the given protein target, rather than making general predictions about aptamer properties or behavior in isolation. This conditioning allows the model to make binding predictions that are specific to each protein-aptamer pair, accounting for their unique molecular interactions and compatibility.

An aptamer refers to a synthetic single-stranded DNA or RNA molecule designed to bind to a specific protein target, whose binding properties our system predicts through computational analysis. Vector representation refers to a machine-readable numerical format that encodes both the primary sequence and chemical properties of aptamers and proteins, serving as input to our neural network system. Embedding refers to the transformed representation produced by our specialized encoders (RNA-FM/Nucleotide Transformer for aptamers, ESM-2 for proteins) that captures complex molecular characteristics relevant to binding prediction. Self-attention network refers to neural network layers that analyze relationships within individual molecule embeddings, identifying important structural and chemical patterns that influence binding potential.

Cross-attention network refers to a neural network component that models potential binding interactions between aptamer and protein pairs by relating their respective embeddings. Prediction score refers to the system's final output indicating binding likelihood, computed by processing combined molecular representations through neural network layers to generate a value between 0 and 1.

### Methods for Binding Prediction and De Novo Aptamer Sequence Generation

### Step (a): Obtaining a Vector Representation of an Aptamer Sequence

In this step, the system transforms a raw aptamer sequence, typically provided as a text string comprising nucleotide bases (adenine, cytosine, guanine, thymine for DNA, or adenine, cytosine, guanine, uracil for RNA), into a machine-readable numerical format suitable for downstream neural network processing. The vectorization process comprises two sub-steps: tokenization and numerical mapping.

During tokenization, the aptamer sequence is parsed into discrete aptamer units. A token may represent an individual nucleotide base or, in alternative embodiments, a k-mer (a subsequence of k consecutive nucleotides) or another chemically meaningful unit. Tokenization preserves the sequential order and chemical identity of the aptamer while segmenting the sequence into manageable computational elements. The choice of tokenization scheme can influence the model's ability to capture local structural motifs and long-range dependencies, but the invention encompasses various tokenization strategies appropriate for oligonucleotide sequences.

Following tokenization, each discrete aptamer unit is mapped to an aptamer numerical vector. This mapping converts each token into a corresponding numerical representation, typically a high-dimensional vector that encodes both the identity of the nucleotide unit and relevant chemical properties such as charge, hydrogen-bonding potential, hydrophobicity, and structural constraints. The numerical vectors enable the machine learning model to process the aptamer sequence while retaining biologically and chemically meaningful information. Various embedding techniques known in the art, including learned embeddings, one-hot encodings, or pretrained nucleotide representations, may be employed to generate these numerical vectors. The resulting vector representation serves as the input for subsequent encoding and embedding generation steps.

### Step (b): Obtaining a Vector Representation of a Molecular Target Protein Sequence

Analogous to step (a), the system converts the molecular target protein sequence—typically provided as a text string comprising amino acid residues in standard one-letter or three-letter code—into a numerical vector representation. This process likewise comprises tokenization and mapping sub-steps tailored for protein sequences.

In the tokenization phase, the protein sequence is segmented into discrete protein units. Each token may correspond to a single amino acid residue or, in alternative implementations, to larger structural or functional motifs such as dipeptides, tripeptides, or residue groups with shared physicochemical properties. Tokenization preserves the linear order of the protein sequence and prepares it for numerical encoding.

Each discrete protein unit is then mapped to a protein numerical vector. The mapping encodes the identity and relevant biochemical characteristics of each amino acid, including hydrophobicity, charge, size, polarity, and secondary structure propensity. These numerical vectors allow the neural network to interpret the protein sequence in a manner consistent with known protein biochemistry and structural biology. Various pretrained protein language models or embedding methods may be used to generate high-quality numerical representations that capture evolutionary, structural, and functional features of the protein sequence. The resulting vector representation forms the input to the protein encoder in step (d).

### Step (c): Generating an Aptamer Embedding Using an Aptamer Encoder

The vector representation of the aptamer sequence obtained in step (a) is processed by an aptamer encoder to generate a high-dimensional aptamer embedding. The aptamer encoder is a specialized neural network component designed to transform the sequential vector representations into a richer, contextualized embedding that captures complex molecular features, including predicted secondary structure, stability, charge distribution, potential binding surfaces, and long-range intramolecular interactions.

In preferred embodiments, the aptamer encoder is implemented as a transformer-based model specifically trained or adapted for nucleic acid sequences. Examples include, but are not limited to, RNA-FM, Nucleotide Transformer, or other architectures capable of processing RNA or DNA sequences. These models leverage self-attention mechanisms to model dependencies between distant nucleotides and to integrate contextual information across the entire sequence. The aptamer embedding produced by the encoder is a set of high-dimensional vectors, one per token, that collectively represent the aptamer's molecular properties in a format optimized for downstream neural network processing.

The aptamer encoder may be pretrained on large corpora of nucleic acid sequences to learn generalizable representations of RNA and DNA structure and function, and may optionally be fine-tuned on aptamer-specific datasets to enhance performance on aptamer-protein binding prediction tasks. The invention is not limited to any particular encoder architecture, and encompasses any neural network model capable of generating meaningful embeddings from aptamer sequence vectors.

### Step (d): Generating a Molecular Target Protein Embedding Using a Protein Encoder

The vector representation of the protein sequence obtained in step (b) is processed by a protein encoder to generate a high-dimensional molecular target protein embedding. Similar to the aptamer encoder, the protein encoder is a specialized neural network—often implemented as a transformer-based protein language model—that converts the sequential protein vectors into a contextualized embedding capturing structural, functional, and interaction-relevant features of the protein.

Suitable protein encoders include, but are not limited to, ESM-2 (Evolutionary Scale Modeling), ProtBERT, ProteinTransformer, or other models pretrained on large-scale protein sequence databases. These encoders learn to represent amino acids in the context of their surrounding sequence, capturing information about secondary structure, solvent accessibility, binding pockets, and evolutionary conservation. The resulting protein embedding is a set of high-dimensional vectors, one per amino acid token, that encode the protein's molecular characteristics in a manner compatible with the aptamer embedding for subsequent interaction modeling.

The protein encoder may be pretrained on diverse protein families and optionally fine-tuned on datasets relevant to aptamer-protein interactions or specific target protein classes. The invention encompasses any protein embedding method capable of generating representations suitable for predicting molecular interactions with aptamers.

### Step (e): Processing the Aptamer Embedding and Molecular Target Protein Embedding Using a Neural Network Comprising Self-Attention and Cross-Attention Networks

In this step, the aptamer embedding from step (c) and the molecular target protein embedding from step (d) are jointly processed by a sophisticated neural network architecture that models both intramolecular relationships (within each molecule) and intermolecular relationships (between the aptamer and the protein).

The self-attention network comprises a plurality of self-attention network layers that independently analyze the aptamer embedding and the protein embedding. Self-attention mechanisms enable the model to identify important relationships within each molecule, such as long-range structural dependencies, critical binding motifs, and context-dependent features. For the aptamer, self-attention captures how different nucleotides interact to form secondary and tertiary structures that influence binding. For the protein, self-attention identifies residues that are spatially or functionally connected, potential binding pockets, and regions of interest for molecular recognition. The self-attention layers produce self-attended embeddings that are enriched with intramolecular contextual information.

The cross-attention network comprises a plurality of cross-attention network layers that model the interactions between the aptamer and protein embeddings. In a typical cross-attention mechanism, the protein embedding serves as the query (Q), while the aptamer embedding provides the key (K) and value (V) matrices (or vice versa in a bidirectional implementation). Cross-attention computes attention scores that quantify the relevance of each aptamer token to each protein token, thereby capturing potential binding interfaces, electrostatic complementarity, shape compatibility, and interaction hotspots. The cross-attention layers produce cross-attended embeddings that encode intermolecular interaction patterns and binding compatibility.

In some embodiments, the architecture employs symmetric or bidirectional cross-attention, wherein both an aptamer-attended protein representation and a protein-attended aptamer representation are computed and subsequently combined. The outputs of the self-attention and cross-attention modules may be summed, concatenated, or otherwise fused to generate a unified representation that integrates both intramolecular features and intermolecular interactions. This combined representation serves as the input to the final prediction module.

The self-attention and cross-attention networks are typically implemented using multi-head attention mechanisms, residual connections, layer normalization, and feedforward neural network sub-layers, following standard transformer architecture principles. The number of layers, attention heads, hidden dimensions, and other architectural hyperparameters may be optimized during training. The invention encompasses various attention-based architectures capable of jointly modeling aptamer and protein sequences for binding prediction.

### Step (f): Generating a Prediction Score Indicating a Likelihood of Binding

The final step generates a prediction score that quantifies the likelihood of binding between the aptamer sequence and the protein molecular target sequence. This score is derived from the outputs of the cross-attention network (and optionally the combined self-attended and cross-attended representations) produced in step (e).

In preferred embodiments, the combined representation is processed through a multi-layer perceptron (MLP), also referred to as a feedforward neural network or fully connected network. The MLP comprises one or more linear transformation layers interleaved with nonlinear activation functions (e.g., ReLU, GELU) and optionally batch normalization or dropout layers. The MLP progressively reduces the high-dimensional combined embedding into lower-dimensional representations and ultimately outputs a single scalar value.

This scalar value is typically transformed into a binding probability score between 0 and 1 using a sigmoid activation function, where values closer to 1 indicate high predicted binding affinity or likelihood, and values closer to 0 indicate low or negligible binding. Alternatively, the score may be interpreted as a continuous binding affinity proxy, a classification logit, or another metric indicative of binding strength or compatibility.

The prediction score enables rapid, high-throughput screening of aptamer-protein pairs without requiring experimental validation or computationally expensive structural simulations. The score incorporates learned patterns from training data comprising known aptamer-protein interactions, binding affinities, and negative examples, and generalizes to novel aptamer and protein sequences not seen during training. The invention contemplates various methods of score generation from attention-network outputs, including but not limited to MLP-based prediction, attention-weighted pooling, and regression heads, provided that the output reflects the predicted binding likelihood between the input sequences.

### Use of Transformer Models for Encoding

The computer-implemented method described above may be further refined by the specification that step (c) comprises using an aptamer transformer model and step (d) comprises using a protein transformer model.

Transformer-based encoder models represent a sophisticated evolution in sequence encoding technology, leveraging self-attention mechanisms to capture long-range dependencies and contextual relationships across entire sequences without the computational limitations of recurrent or convolutional approaches. An aptamer transformer model is a neural network architecture specifically designed or adapted for encoding nucleotide sequences, including DNA and RNA aptamers. Representative examples include the Nucleotide Transformer (variants such as Nucleotide Transformer-500M or Nucleotide Transformer-2.5B), RNA-FM (RNA Foundation Model), or other transformer-based models pretrained on large corpora of genomic and transcriptomic sequences. These models have been trained on diverse genetic material, enabling them to learn generalizable patterns of nucleotide composition, secondary structure determinants, and sequence motifs that are predictive of molecular properties and function.

The aptamer transformer model processes the tokenized aptamer vectors through multiple layers of multi-head self-attention and feedforward sublayers, progressively refining the representation to capture both local nucleotide interactions (e.g., base-pairing patterns, stacking forces) and global structural features (e.g., hairpins, bulges, multi-stem conformations). The output is a contextualized embedding for each token in the aptamer sequence, wherein each nucleotide's representation encodes information about its chemical identity, its predicted local environment (secondary structure), and its role within the overall three-dimensional molecular structure. Because transformer models employ attention mechanisms, they can weight the importance of distant nucleotides, allowing the model to recognize aptamer binding motifs that span non-contiguous regions of the sequence, a capability that is particularly important for aptamers, which often form complex tertiary structures bridging their primary sequence.

Similarly, a protein transformer model is a neural network architecture specifically designed for encoding amino acid sequences in proteins. Representative examples include ESM-2 (Evolutionary Scale Modeling, versions ranging from ESM-2-8M to ESM-2-15B), ProtBERT, ProteinTransformer, or other transformer-based protein language models. These models are typically pretrained on large multiple sequence alignments (MSAs) of homologous proteins or on entire reference protein databases, enabling them to learn evolutionary relationships, structural properties, and functional conservation patterns. A protein transformer model processes tokenized protein vectors through self-attention layers, generating contextualized embeddings that capture the chemical properties of each amino acid (charge, hydrophobicity, size), its evolutionary conservation across species, its predicted secondary structure context (alpha-helix, beta-sheet, coil), its likely solvent accessibility, and its functional role within known protein domains or families.

By employing both an aptamer transformer and a protein transformer, the method achieves several advantages: (1) specialized feature extraction, wherein each encoder is optimized for the unique chemistry, structure, and functional properties of its respective molecule type; (2) pretrained knowledge, wherein the encoders inherit learned representations from large-scale biological data, reducing the need for task-specific training data; (3) improved contextual understanding, wherein long-range dependencies and global molecular architecture are captured by attention mechanisms; and (4) compatibility with downstream processing, wherein both aptamer and protein embeddings are in a shared high-dimensional space, enabling direct comparison and interaction modeling in the cross-attention network of step (e).

### Analysis of Internal and Intermolecular Relationships

The computer-implemented method may be further elaborated in that step (e) comprises (i) analyzing internal relationships within the aptamer embedding and within the molecular target protein embedding using the self-attention network, and (ii) modeling interactions between the aptamer embedding and the molecular target protein embedding using the cross-attention network.

The self-attention network performs an in-depth analysis of intramolecular relationships by computing attention weights that quantify how each token in a sequence relates to all other tokens in the same sequence. For the aptamer embedding, self-attention layers identify nucleotides that are spatially or functionally coupled, even if they are distant in the primary sequence.

For the protein embedding, self-attention similarly identifies amino acid residues that interact through hydrogen bonds, hydrophobic contacts, disulfide bridges, or spatial proximity within the folded protein structure. Self-attention captures which residues form the core hydrophobic interior, which residues are exposed on the surface and likely to participate in binding, and which residues are conserved across evolution and therefore functionally critical.

In preferred embodiments, multi-head self-attention is employed, wherein the self-attention computation is performed in parallel across multiple sets of learned Q, K, V projections (e.g., 8 attention heads). Each head learns to focus on different relationships—for example, one head might specialize in capturing short-range interactions while another captures long-range structural dependencies. The outputs of all heads are concatenated and linearly transformed, yielding a richer, multi-faceted representation of intramolecular relationships. This multi-head mechanism enhances the model's expressiveness and robustness.

The cross-attention network, by contrast, explicitly models intermolecular interactions, that is, the binding relationships between aptamer and protein. In a cross-attention layer, the embeddings from both molecules are processed asymmetrically: in one configuration, the protein embedding is projected to form query (Q) tokens, while the aptamer embedding is projected to form key (K) and value (V) tokens. The attention mechanism then computes cross-attention scores.

These cross-attention scores quantify the relevance of each aptamer nucleotide to each protein residue, thereby identifying potential contact points and binding interfaces. Numerically high attention weights indicate predicted molecular contacts or electrostatic/hydrogen-bonding compatibility. In some embodiments, the cross-attention computation is performed bidirectionally, computing both protein-to-aptamer and aptamer-to-protein attention patterns. This bidirectional approach captures the binding interface from both molecular perspectives and can reveal asymmetries in binding, for example, cases where a large surface area of the aptamer contacts a small pocket on the protein, or vice versa.

In multi-layer configurations (embodied in the phrase “plurality of cross-attention network layers”), successive cross-attention layers progressively refine the representation of intermolecular interactions. Early layers may capture gross geometric and electrostatic complementarity, while deeper layers capture more nuanced interaction patterns, allosteric effects, and conformational changes induced by binding. The accumulation of information across multiple layers generates a sophisticated, hierarchical model of how the aptamer and protein molecules are predicted to interact at the molecular level.

### Multi-Layer Perceptron Processing for Final Score Generation

In embodiments, step (f) may comprise processing the outputs of the cross-attention network of step (e) through a multi-layer perceptron (MLP) to produce a value between 0 and 1.

Following the self-attention and cross-attention processing, the combined aptamer and protein representations (which may be summed, concatenated, or otherwise fused) are passed through a multi-layer perceptron to generate the final binding prediction score. A multi-layer perceptron is a feedforward neural network comprising successive layers of neurons (or computing units), each connected to all neurons in the subsequent layer. Each layer performs an affine transformation (a weighted sum followed by an additive bias), followed by a nonlinear activation function.

In a representative implementation, the MLP architecture might comprise:
a. Input layer: A high-dimensional vector combining the cross-attended and self-attended aptamer and protein representations (e.g., dimension 1024 or 1280).
b. First hidden layer: An affine transformation reducing the dimensionality to a smaller intermediate size (e.g., 512 neurons), followed by a nonlinear activation such as ReLU (Rectified Linear Unit), which applies the function f(x)=max(0,x), or GELU (Gaussian Error Linear Unit). This nonlinearity enables the MLP to model complex, non-linear relationships between the input features and the binding outcome.
c. Optional intermediate layers: Additional hidden layers (e.g., a second hidden layer with 256 neurons), again followed by activation functions. These additional layers increase the representational capacity of the MLP, allowing it to capture increasingly abstract and complex patterns.
d. Regularization: Between layers, techniques such as batch normalization (normalizing the distribution of activations to have zero mean and unit variance) or dropout (randomly zeroing a fraction of activations during training to prevent overfitting) may be applied to improve generalization.
e. Output layer: A single neuron with a sigmoid activation function, defined as σ(x)=1/(1+e\hat{ }(−x)), which transforms the final hidden representation into a value strictly between 0 and 1. This output is interpreted as the binding probability score or binding likelihood.

The sigmoid activation is particularly appropriate for binary classification tasks (bound vs. not bound). A score close to 1 indicates high predicted affinity or likelihood of binding, while a score close to 0 indicates low or negligible binding. The continuous nature of the score (rather than a discrete binary classification) allows for ranking and prioritization of aptamer candidates—a critical feature for high-throughput screening and aptamer library analysis.

During training, the MLP parameters (weights and biases) are optimized to minimize a loss function such as binary cross-entropy. This loss function penalizes predictions that diverge from experimental ground truth, driving the MLP to learn a mapping from aptamer-protein representations to accurate binding predictions.

### Identification of Critical Binding Residues and Nucleotides (Interpretability)

A step (g) may be further added to the method, namely comprising identifying a specific residue on the molecular target protein sequence with the highest contribution to the predicted binding interaction, and identifying a specific nucleotide on the aptamer sequence with the highest contribution to the predicted binding interaction.

This interpretability step leverages the attention mechanisms inherent in the neural network to provide mechanistic insights into predicted binding interactions. The cross-attention layers produce attention weight matrices that directly encode which aptamer nucleotides are predicted to interact most strongly with each protein residue, and vice versa. These attention weights can be visualized and analyzed to identify critical binding residues and critical nucleotides.

### Extraction of Attention Weights

The attention weight matrix from a cross-attention layer, after softmax normalization, is a matrix A∈R\hat{ }(M×N), where M is the number of protein tokens and N is the number of aptamer tokens. Element A_ij represents the normalized attention weight linking protein token i to aptamer token j. High values in A indicate predicted molecular contacts or compatibility.

### Aggregation Across Layers and Heads

In multi-layer and multi-head architectures, multiple attention weight matrices are generated. These can be aggregated by averaging across layers and attention heads to yield a single consensus attention matrix A⁻, which provides a stable estimate of the overall predicted interaction pattern.

### Visualization and Validation

The attention-derived interaction map can be visualized as a heatmap or network diagram, showing which residues are predicted to contact which nucleotides. This visualization serves as a computational analog of crystal structure data or mutagenesis experiments in the experimental literature, providing a testable prediction of the binding interface. Experimental biologists can then prioritize mutagenesis studies, alanine scans, or structural validation (via NMR, cryo-EM, or molecular dynamics) to confirm or refine the computationally predicted interface.

In embodiments, this interpretability layer enables explainable artificial intelligence (XAI) for molecular discovery, allowing researchers to understand not merely the predicted affinity but also the molecular basis of binding—a critical requirement for regulatory approval, mechanistic validation, and rational optimization of aptamer and protein sequences.

### Target-Conditioned Candidate Aptamer Prediction Apparatus (Base Apparatus)

The present disclosure includes a target-conditioned candidate aptamer prediction apparatus, which is a comprehensive computer system and hardware architecture specifically designed to perform high-throughput, AI-driven prediction of aptamer-protein binding interactions. The apparatus comprises interconnected hardware and software components that work in concert to receive molecular sequences, process them through specialized neural networks, and output quantitative binding predictions.

### Processor Unit

The processor unit is the computational engine of the apparatus. In contemporary implementations, the processor unit may comprise one or more central processing units (CPUs) and/or graphics processing units (GPUs). CPUs are well-suited for general-purpose computation and control flow, including data loading, sequence tokenization, and model management. GPUs, by contrast, are highly optimized for the parallel, matrix-multiplication-intensive operations that dominate transformer-based neural networks and attention mechanisms. In preferred embodiments, the processor unit includes NVIDIA GPUs (e.g., Tesla A100, H100, or consumer variants such as RTX 4090) or TPUs (Tensor Processing Units), which provide throughput in the range of teraFLOPS (trillion floating-point operations per second), enabling the rapid processing of thousands of aptamer-protein pairs per minute.

The processor unit executes the neural network forward pass (inference) to generate binding predictions, and during training phases, executes the backward pass to compute gradients and optimize model parameters. The processor unit may also execute preprocessing operations such as sequence alignment, motif extraction, and attention weight aggregation for interpretability analysis.

### Memory Unit and Machine Learning Model Storage

The memory unit comprises both volatile memory (RAM, typically 32 GB to 512 GB in high-performance configurations) and persistent storage (solid-state drives, network-attached storage, or cloud storage), and is responsible for storing the machine learning model along with associated data structures and intermediate representations.

The machine learning model stored in the memory unit comprises the following sub-components:
a. Vectorization Component: The vectorization component is a software module responsible for converting raw aptamer and protein sequences (typically provided as text strings or FASTA-formatted files) into numerical vector representations suitable for neural network processing. This component implements the tokenization and embedding mapping operations described in dependent claims 2 through 5. The vectorization component may employ pretrained nucleotide embedding tables (e.g., learned representations from nucleotide language models) and amino acid embedding tables (e.g., physicochemical properties or evolutionary embeddings). In some implementations, the vectorization component includes lookup tables or hash maps that map tokens to their corresponding numerical vectors, enabling rapid conversion of sequences into embedding space. The vectorization component is configured to handle variable-length sequences, padding or truncating as necessary to match the model's expected input dimensions.
b. Aptamer Encoder: The aptamer encoder is a transformer-based or recurrent neural network module specifically designed to process nucleotide embeddings and generate contextualized aptamer embeddings. The aptamer encoder comprises multiple layers of multi-head self-attention and feedforward sublayers, progressively refining nucleotide representations to capture secondary structure, tertiary interactions, and binding motifs. The parameters of the aptamer encoder (attention weights, feedforward weights, layer normalization gains and biases) are stored in the memory unit. The aptamer encoder may be pretrained on large nucleic acid corpora and either frozen or fine-tuned during task-specific training. The encoder's output is a sequence of high-dimensional vectors (one per nucleotide token), collectively forming the aptamer embedding.
c. Molecular Protein Target Encoder: Analogous to the aptamer encoder, the molecular protein target encoder is a specialized neural network module configured to process amino acid embeddings and generate contextualized protein embeddings. The protein encoder comprises transformer layers optimized for amino acid sequences, incorporating knowledge of protein structure, evolutionary conservation, and biochemical properties. The protein encoder outputs a sequence of high-dimensional vectors (one per amino acid token), collectively forming the molecular target protein embedding.
d. Self-Attention Network: The self-attention network is a collection of self-attention layers configured to independently analyze the aptamer embedding and the protein embedding. The self-attention network contains learnable parameters, including query (Q), key (K), and value (V) projection matrices for each attention head, layer normalization parameters, and feedforward network weights. During operation, the self-attention network processes each embedding separately, generating self-attended embeddings enriched with intramolecular contextual information. The self-attention network does not directly model interactions between the aptamer and protein; rather, it refines each molecule's internal representation.
e. Cross-Attention Network: The cross-attention network is a set of cross-attention layers configured to explicitly model interactions between aptamer and protein embeddings. The cross-attention network contains separate Q, K, V projection matrices for each molecule, allowing asymmetric or bidirectional attention patterns. In a representative configuration, protein embeddings are projected to query (Q) tokens, while aptamer embeddings are projected to key (K) and value (V) tokens, enabling the network to compute which aptamer nucleotides are most relevant to each protein residue. The cross-attention network outputs combined representations (fused aptamer-protein embeddings) that encode intermolecular binding relationships. These combined representations serve as input to downstream prediction or generation modules.

### Interdependency and Data Flow

The components of the machine learning model are connected in a sequential data pipeline: raw sequences→vectorization component→aptamer and protein encoders (in parallel)→self-attention network→cross-attention network→(optionally) multi-layer perceptron→binding prediction score or further processing.

### Nucleic Acid Encoder for DNA Aptamer Embedding

The machine learning model further comprises a nucleic acid encoder for DNA aptamer embedding. While the base apparatus references an “aptamer encoder” without specifying whether it is optimized for DNA or RNA, some embodiments explicitly include a dedicated DNA aptamer encoder.

In practice, nucleotide sequences can be RNA (containing adenine, cytosine, guanine, uracil) or DNA (containing adenine, cytosine, guanine, thymine). Although the chemical difference between RNA and DNA is subtle (a single 2′-OH group on the ribose sugar), their biological roles and structural properties differ significantly. RNA aptamers fold into compact three-dimensional structures stabilized by extensive base-pairing and non-canonical interactions, whereas DNA aptamers typically adopt sparser, more extended conformations due to their greater rigidity.

A nucleic acid encoder for DNA aptamer embedding is a specialized transformer or recurrent neural network trained specifically on DNA sequences, allowing it to capture DNA-specific structural features, thermodynamic stability patterns, and binding properties. Such an encoder might be trained on large collections of genomic DNA, SELEX-derived DNA aptamer libraries, or synthetic DNA aptamer datasets.

In embodiments, the apparatus comprises both an RNA aptamer encoder and a DNA aptamer encoder, enabling the model to handle both RNA and DNA inputs with appropriate specialized processing. The choice of encoder can be made at runtime based on the input sequence type, or both encoders can be applied in parallel with subsequent fusion or selection logic.

The inclusion of a dedicated DNA aptamer encoder enhances the apparatus's capability to accurately model DNA-aptamer-protein interactions, which are of significant commercial and therapeutic interest (e.g., aptamers used in diagnostic assays, therapeutics, and bioanalytical applications often employ DNA due to its greater chemical stability and lower cost compared to RNA).

### Training Unit for Parameter Optimization

The apparatus may include a training unit configured to optimize parameters of the machine learning model to fit experimental binding data quantified via a convex, differentiable loss function.

The training unit is a software component (and potentially associated hardware accelerators) dedicated to supervised learning of the machine learning model. The training unit may perform the following operations:

Data Loading and Preprocessing: The training unit receives a dataset comprising pairs of (aptamer sequence, protein sequence, experimental binding label or affinity value). The labels are quantitative measures such as dissociation constant (K_d), binding affinity (AG), or binary classification (bound/not bound). The training unit preprocesses sequences, vectorizes them using the vectorization component, and organizes them into mini-batches suitable for gradient-based optimization.

Forward Pass: For each mini-batch, the training unit executes the complete model pipeline—vectorization, encoding, self-attention, cross-attention, and (optionally) MLP prediction—to generate predicted binding scores for all pairs in the batch.

Loss Computation: The predicted binding scores are compared to experimental ground truth using a convex, differentiable loss function. A convex loss function has the property that any local minimum is also a global minimum, ensuring that gradient-based optimization converges reliably.

Backpropagation: The loss is backpropagated through the entire neural network using the chain rule, computing gradients ∂L/∂θ for all model parameters θ (weights, biases, projection matrices).

Parameter Updates: Gradients are used to update model parameters via an optimization algorithm such as stochastic gradient descent (SGD), Adam, or AdamW.

Validation and Early Stopping: Periodically, the training unit evaluates the model on a held-out validation set using metrics such as area under the receiver-operating-characteristic curve (AUROC), precision-recall balance, or Pearson correlation between predicted and experimental affinities. If validation performance plateaus or decays, training is stopped early to prevent overfitting.

Regularization: To prevent overfitting, the training unit may employ L2 regularization (weight decay), dropout, or data augmentation (e.g., introducing noise into sequences).

By employing a convex, differentiable loss function, the training unit guarantees that optimization will find a low-loss solution, enabling the model to learn from experimental data and improve its binding predictions over time.

### Critical Motif Storage and Retrieval

In embodiments, the memory unit is configured to store representations of critical motifs for protein target-binding, wherein representations of critical motifs for protein target-binding are adapted to be referenced and arranged when prompted using a novel protein sequence.

A critical motif is a short sequence element (typically 3-15 nucleotides or amino acids) that recurs across multiple known aptamer-protein pairs and is predictive of binding affinity. Critical motifs encode recurring interaction patterns—for example, a specific DNA sequence that frequently contacts a conserved protein binding pocket, or a protein domain fold that frequently interacts with aptamers bearing a particular structural feature.

The apparatus includes a motif database stored in the memory unit. This database comprises:
a. Motif Sequences: Canonical or consensus sequences for aptamer motifs (e.g., GGGAG for G-quadruplex formation) and protein motifs (e.g., Zn-finger domains, RGG-rich regions).
b. Motif Embeddings: Vectorized representations of motifs in the same embedding space as the main model, enabling rapid similarity matching and retrieval.
c. Binding Statistics: For each motif, metadata such as frequency in known aptamer-protein pairs, average affinity contribution, and conservation across different protein families.
d. Motif Retrieval and Arrangement Logic: When prompted with a novel protein sequence, the apparatus performs the following operations:
   i. Motif Scanning: The apparatus scans the novel protein sequence for the presence of known critical protein motifs, generating a motif profile.
   ii. Complementary Aptamer Motif Prediction: Based on the detected protein motifs, the apparatus predicts which aptamer motifs are most likely to bind strongly, using prior statistics or learned associations from training data.
   iii. Ranking and Arrangement: The predicted aptamer motifs are ranked by predicted binding affinity contribution and arranged in an optimal sequence (e.g., separated by linker regions to avoid steric hindrance).
   iv. Sequence Suggestion: The apparatus outputs candidate aptamer sequences incorporating the predicted motifs, providing users with interpretable, knowledge-grounded sequence suggestions.

This motif-driven approach bridges the gap between black-box neural network predictions and interpretable, biologically grounded design principles. Users can understand and validate the model's suggestions by recognizing known binding motifs and their empirically validated contribution to affinity.

### Multi-Layer Perceptron for Binding Prediction Score

The apparatus further comprises a multi-layer perceptron configured to generate a binding prediction score.

As described previously, the multi-layer perceptron (MLP) is the final prediction layer that converts the combined cross-attention embeddings into a scalar binding prediction score. In the apparatus context, the MLP is a persistent, stored component of the machine learning model.

The MLP comprises:
a. Input Projection: A learnable linear transformation that projects the combined embeddings (typically concatenated or summed aptamer and protein representations after cross-attention) from their high-dimensional space (e.g., 1280 dimensions) to a smaller hidden dimension (e.g., 512 dimensions).
b. Hidden Layers: One or more fully connected layers with nonlinear activations (ReLU, GELU, etc.), progressively reducing dimensionality and learning increasingly abstract features predictive of binding.
c. Dropout and Batch Normalization: Regularization layers that reduce overfitting and stabilize training.
d. Output Layer: A final linear layer projecting to a single scalar, followed by a sigmoid activation to produce a score in the range [0,1].

The MLP is trainable via the training unit and optimizable to fit experimental data. Storing the MLP as a distinct apparatus component highlights its critical role in the binding prediction pipeline and enables claims directed specifically to MLP-based prediction systems.

### Iterative Sequence Optimization

In further embodiments, the processor unit is configured to iteratively modify the aptamer sequences to optimize predicted binding scores.

This claim captures the apparatus's capability to perform in silico optimization of aptamer sequences without requiring experimental synthesis or validation. The iterative modification process comprises:
a. Sequence Initialization: Start with an initial aptamer sequence (provided by the user, randomly generated, or sampled from a library).
b. Scoring: Compute the binding prediction score for the initial sequence using the machine learning model.
c. Modification: Apply a small perturbation to the sequence—for example, substituting a single nucleotide, inserting a nucleotide, or deleting a nucleotide. Alternatively, apply more sophisticated modifications such as local sequence swaps, mutation of predicted high-contact nucleotides, or incorporation of known binding motifs.
d. Re-scoring: Compute the binding prediction score for the modified sequence.
e. Acceptance Criterion: If the modified score is higher (indicating stronger predicted binding), accept the modification and continue from the new sequence. If the score is lower, reject the modification and try a different perturbation.
f. Iteration: Repeat the modification-scoring-acceptance cycle for a specified number of iterations or until convergence (no further improvements detected).

Optimization Algorithms: The processor unit may implement various optimization algorithms tailored to this discrete sequence optimization problem:
a. Greedy Local Search: At each step, evaluate all single-nucleotide mutations and select the one with the highest predicted affinity.
b. Simulated Annealing: Allow occasional acceptance of worse scores to escape local optima.
c. Genetic Algorithms: Maintain a population of sequences and perform selection, crossover, and mutation operations to evolve high-affinity sequences.
d. Reinforcement Learning: Treat sequence design as a Markov decision process, training an agent to select mutations that maximize cumulative binding scores.
e. Monte Carlo Tree Search (MCTS): Recursively expand a tree of sequence modifications, using upper confidence bounds to balance exploration and exploitation.

This iterative optimization capability enables rapid, computationally efficient screening of vast sequence spaces (on the order of 4\hat{ }L sequences for DNA of length L), generating optimized aptamer candidates for experimental validation without the cost and time of traditional SELEX or directed evolution experiments.

### Input/Output Module and Binding Interface Visualization

The apparatus may further comprise an input/output (I/O) module configured to receive sequence inputs and provide visualization of predicted binding interfaces.

The I/O module serves as the user-facing interface to the apparatus, handling data ingress and output visualization:

### Sequence Input:

a. Text Input: Users enter aptamer or protein sequences as plain text strings or FASTA-formatted files.
b. Database Queries: The I/O module retrieves sequences from external databases (e.g., UniProt for proteins, NCBI GenBank for nucleic acids, or custom aptamer libraries).
c. Batch Processing: Users upload multi-sequence files (CSV, FASTA, Excel) for high-throughput analysis of many aptamer-protein pairs simultaneously.
d. Parameter Specification: Users specify additional options such as optimization algorithm, number of iterations, motif preferences, or confidence thresholds.

### Binding Interface Visualization:

The I/O module generates visual representations of predicted binding interfaces, including:

Attention Heatmaps: Two-dimensional heatmaps displaying the cross-attention weight matrix, with aptamer nucleotides on one axis and protein residues on the other. Color intensity indicates predicted interaction strength. This provides a direct visual summary of which nucleotides contact which residues.

Network Diagrams: Graph-based visualizations showing aptamer nucleotides and protein residues as nodes, with edges connecting predicted interaction pairs. Edge thickness or color can encode attention weights or predicted contribution to binding energy.

Sequence Alignment Displays: Aptamer and protein sequences are displayed in parallel, with predicted contact residues/nucleotides highlighted or annotated. Arrows or connection lines link interacting pairs.

3D Structure Visualization (if structural models are available): The apparatus can overlay predicted binding interfaces onto known or predicted three-dimensional structures of the protein and aptamer, providing spatial context for the interactions.

Motif Annotation: Predicted critical motifs are highlighted within the sequences and annotated with binding statistics.

Scoring Reports: Tabular and graphical summaries of binding predictions, including:
a. Point estimates of affinity (e.g., K_d predictions in nM or μM).
b. Confidence intervals or Bayesian posterior distributions over affinities.
c. Ranking of sequences by predicted affinity (useful for prioritizing candidates for synthesis).
d. Sensitivity analysis: how much the prediction changes if individual nucleotides or residues are mutated.

Output Formats: The I/O module exports results in multiple formats suitable for downstream analysis:
a. CSV/Excel: Tabular data for sorting, filtering, and statistical analysis.
b. JSON: Structured data suitable for programmatic access and integration into external pipelines.
c. PDF/PNG: Publication-quality figures for reports and papers.
d. Interactive HTML: Web-based dashboards enabling exploratory browsing of results.

The I/O module transforms the apparatus from a research tool into a practical, user-friendly system suitable for deployment in academic research, pharmaceutical development, and diagnostic device design.

### Core Training Procedure for Aptamer-Protein Binding Prediction

The fundamental method of training a machine learning model for predicting aptamer-protein target binding is now described in greater detail. This method encapsulates the supervised learning framework that transforms a randomly initialized or pretrained neural network into a high-performance binding prediction system by exposing it to empirical binding data and optimizing its parameters via gradient descent.

### Step (a): Curation of Training and Benchmark Datasets

The first step involves curating a training dataset and a benchmark dataset comprising known and proprietary aptamer-protein target pairs and corresponding binding data, in addition to known natural oligonucleotide-target complexes.

Effective machine learning requires large, high-quality, diverse datasets. The curation process comprises:

Training Dataset Assembly: A comprehensive collection of aptamer-protein target pairs for which experimental binding measurements are available. This dataset may comprise:
a. Published aptamer selections: Sequences from peer-reviewed literature describing SELEX, in vitro display, or high-throughput screening experiments, together with reported dissociation constants (K_d), inhibition constants (K_i), or other affinity measures.
b. Proprietary datasets: Internal experimental data from the organization developing the model, including aptamers generated through the organization's own selection campaigns and measured against internal protein targets.
c. Commercial databases: Curated collections such as the Aptamer Database (ApDB), which aggregates published aptamers and their properties.
d. Natural oligonucleotide-target complexes: In addition to synthetic aptamers, the training data may include natural RNA-protein interactions (e.g., ribosomal RNA binding to ribosomal proteins, regulatory RNAs binding to transcription factors) and natural DNA-protein interactions (e.g., transcription factor binding sites, nucleosome positioning sequences). These natural complexes provide valuable training signal because they represent evolutionarily optimized, functional interactions and encode deep biological constraints and principles.

Binding Data Standardization: Raw binding measurements from different experimental sources are standardized to a common metric. For example, K_d values are preferred over K_i or IC₅₀ values (when available), and measurements are converted to consistent units (e.g., nM) and temperature conditions. Inconsistent or outlier measurements are flagged and potentially excluded.

Benchmark Dataset Preparation: A held-out subset of the data (typically 10-20% of the total) is reserved as a benchmark or validation set. This benchmark set is not used during training and serves to estimate the model's generalization performance on unseen data. The benchmark set should be representative of the diversity of sequences and binding affinities in the training set, to provide an honest estimate of real-world performance.

Data Diversity and Balance: The curation process ensures that the dataset is diverse with respect to:
a. Protein families: The dataset includes diverse protein targets (e.g., transcription factors, enzymes, membrane proteins, antibodies, viral proteins) to prevent the model from overfitting to sequence features specific to one protein class.
b. Aptamer lengths and sequences: Aptamers ranging from short ~15 nucleotide aptamers to longer ~80 nucleotide aptamers are included, and the nucleotide composition should span GC-rich, AT-rich, and balanced sequences.
c. Binding affinities: The dataset includes strong binders (K_d in the picomolar to nanomolar range), moderate binders (low micromolar), and weak or non-binding pairs, ensuring the model learns to distinguish across the full affinity spectrum.
d. Binding mechanisms: The dataset encompasses direct recognition (aptamer binds directly to protein), indirect binding (aptamer stabilizes protein conformations or promotes oligomerization), and non-binding negative controls.

### Step (b): Generating Vector Representations of Aptamer-Protein Target Pairs

In this step, the system generates a vector representation of at least one aptamer-protein target pair. This vectorization is performed for all pairs in the training and benchmark datasets.

As described previously, vectorization comprises:
a. Aptamer sequence tokenization: The aptamer sequence is segmented into discrete nucleotide tokens (or k-mers).
b. Aptamer embedding mapping: Each nucleotide token is mapped to a high-dimensional numerical vector encoding its chemical identity and properties.
c. Protein sequence tokenization: The protein sequence is segmented into amino acid tokens (or motif tokens).
d. Protein embedding mapping: Each amino acid token is mapped to a high-dimensional vector encoding its identity and biochemical properties.

The vectorized pairs are batched together for efficient parallel processing on GPUs. A typical batch might comprise 32-256 aptamer-protein pairs.

### Step (c): Generating Aptamer-Protein Embeddings

The vector representations are processed through the aptamer encoder and protein encoder (as described in earlier claims) to generate contextualized embeddings. Specifically:
a. The aptamer vector representation is passed through the aptamer transformer encoder, producing an aptamer embedding, a sequence of high-dimensional vectors (one per nucleotide) that captures the predicted structure and functional properties of the aptamer.
b. The protein vector representation is passed through the protein transformer encoder, producing a protein embedding, a sequence of high-dimensional vectors (one per amino acid) that encodes the protein's structure, evolutionary context, and functional properties.

At this stage in the training pipeline, the encoder parameters may be frozen (not updated during training, if the encoders are pretrained and assumed to be generally applicable) or trainable (updated during training, if the encoders are being jointly fine-tuned for the specific binding prediction task).

### Step (d): Processing Through Self-Attention and Cross-Attention Networks

The aptamer embedding and protein embedding are jointly processed through the self-attention network and cross-attention network as described in dependent claim 3. This processing generates:
a. Self-attended representations: Enriched embeddings capturing intramolecular relationships within the aptamer and protein.
b. Cross-attended representations: Embeddings encoding intermolecular interactions and binding interfaces between the aptamer and protein.
c. Combined representation: A fused embedding (via summation, concatenation, or learned fusion) combining both self-attended and cross-attended information.
   Step (e): Comparing Predicted Binding Scores with Experimental Binding Data

The combined representation is processed through the multi-layer perceptron (MLP) to generate a predicted binding score y\hat{ }—a scalar value in the range [0,1] representing the model's estimate of binding likelihood or affinity.

### Step (f): Updating Model Parameters via Gradient-Based Optimization

In the final step, at least one parameter of the machine learning model is updated using gradient-based optimization, with the goal of minimizing aptamer-protein binding prediction error.

This process comprises:

Backpropagation: The loss is backpropagated through the entire neural network, computing gradients ∂L/∂θ for all trainable parameters θ (weights, biases, and other learned quantities in the aptamer encoder, protein encoder, self-attention network, cross-attention network, and MLP).

Iterative Training: The process of vectorization, embedding generation, forward pass, loss computation, and backpropagation is repeated for many mini-batches over multiple epochs (passes through the entire training dataset). Typical training runs 10-100 epochs, depending on dataset size and model complexity.

Convergence Monitoring: During training, the batch loss and validation loss (computed on the benchmark dataset, without updating parameters) are continuously monitored. Training continues until convergence is detected (loss plateaus) or a maximum number of epochs is reached.

By iteratively applying gradient-based updates, the model's parameters are adapted to minimize prediction error on the training data, and ideally, to generalize to unseen test data.

### Inclusion of Negative Examples

The training dataset comprises at least one negative example of a non-binding aptamer-protein target pair.

Negative examples, instances where no binding occurs, are critical for training robust classifiers or regressors. Without negative examples, the model could learn a trivial solution (e.g., predicting high affinity for all inputs, which would achieve high accuracy if the training set is dominated by binders).

Negative examples in the aptamer context include:
a. Experimentally validated non-binders: Aptamers that were screened against a protein target and did not bind, documented in published literature or internal experiments.
b. Mismatched pairs: Randomly paired aptamer and protein sequences that are not known to interact. These serve as “control” negative examples and represent the base rate of non-binding.
c. Aptamers selected against other targets: Aptamers known to bind protein A, when paired with protein B (which was not the original target), often do not bind protein B. These provide realistic negative examples of target-specificity failures.
d. Sequence-scrambled or mutated aptamers: Variants of known binders, mutated or shuffled to destroy binding, providing negative examples with sequence similarity to true binders.

The inclusion of balanced negative examples (e.g., a 1:1 or 1:3 ratio of positives to negatives) ensures the model learns to discriminate binders from non-binders and does not suffer from class imbalance bias.

### Backpropagation Through all Layers

Step (f) further comprises a step of using backpropagation through the self-attention network, the cross-attention network, and an encoder used for generating the aptamer-protein embedding of step (c).

This claim emphasizes that the gradient-based optimization is performed end-to-end, updating not only the downstream MLP but also the intermediate self-attention and cross-attention layers, and critically, the aptamer and protein encoders.

Full Backpropagation Through Encoders: In principle, the gradients flow all the way back through the transformer encoders to the vector representation layer. This allows the encoders to be fine-tuned on the specific binding prediction task, even if they were pretrained on general nucleotide or protein modeling tasks.

For example, a pretrained aptamer encoder (trained on predicting the next nucleotide in a random RNA sequence) may learn very general structural features. However, by backpropagating gradients from the binding prediction loss, the encoder's attention patterns and feedforward weights can be specialized to emphasize features predictive of aptamer-protein binding—for instance, exposing or hiding specific nucleotides that are known to interact with cognate protein targets.

Selective Fine-Tuning: In some embodiments, only certain layers of the encoders are fine-tuned, while early layers remain frozen. This approach, sometimes called layer-wise fine-tuning, leverages the general features learned during pretraining (e.g., base-pairing patterns, secondary structure) while allowing task-specific specialization in deeper layers.

Computational Efficiency: Full backpropagation through all layers increases computational cost (memory consumption and time per training iteration) compared to freezing encoders. However, it can yield significant performance improvements if the pretraining task and the binding prediction task are sufficiently different.

### Validation on Held-Out Test Set

The training method further comprises a step (g) of validating machine learning model performance using a held-out test set of aptamer-protein target pairs.

Test Set Validation is a critical practice in machine learning to assess generalization performance—that is, how well the model performs on data it has never seen during training.

Test Set Preparation: A held-out test set (typically 10-20% of the total data, disjoint from both the training and benchmark sets) is reserved exclusively for final evaluation. The test set should be representative of the diversity of the full dataset and should ideally include some pairs not seen in the benchmark set.

Validation Metrics: The model is evaluated on the test set using metrics appropriate to the task:
a. For binary classification (bound/not bound):
b. Accuracy: Fraction of predictions that are correct.
c. Precision: Among predicted positives, fraction that are true positives.
d. Recall (Sensitivity): Among true positives, fraction that are correctly predicted.
e. F1-Score: Harmonic mean of precision and recall, balancing both metrics.
f. AUROC (Area Under the Receiver-Operating-Characteristic Curve): Probability that the model ranks a random positive higher than a random negative.
g. AUPRC (Area Under the Precision-Recall Curve): Relevant for imbalanced datasets.
h. For continuous affinity prediction:
i. Pearson correlation: Linear correlation between predicted and experimental affinities.
j. Spearman correlation: Rank correlation, robust to outliers.
k. MSE (Mean Squared Error) or MAE (Mean Absolute Error): Magnitude of prediction errors.
l. Normalized RMSE: Root mean squared error normalized to the range of experimental values.

Generalization Assessment: If test performance is significantly worse than training performance, overfitting is suspected. Overfitting occurs when the model has memorized noise or specific features of the training set rather than learning generalizable patterns. Mitigation strategies include regularization (dropout, L2), early stopping, or data augmentation.

Hyperparameter Tuning: The benchmark set may be used to perform hyperparameter tuning—adjusting learning rate, batch size, number of layers, dropout probability, etc.—to optimize validation performance. The test set is then used to provide an unbiased estimate of final performance.

Binding Affinity Measurements from Experiments

The training dataset comprises binding affinity measurements obtained through experimental methods.

### Reference to Specific SEQ IDs and Experimental Affinity Data

The training dataset comprises experimentally-measured binding affinities for aptamers corresponding to SEQ ID NO:1, SEQ ID NO:2, SEQ ID NO:4, SEQ ID NO:17, or SEQ ID NO:18, wherein the experimentally-measured binding affinities serve as labeled training data for optimizing the machine learning model.

The invention creates a specific, concrete linkage between the patent application's Sequence Listing and the training data used to optimize the machine learning model.

Sequence Listing Integration: The patent application includes a Sequence Listing XML file (referenced in the cross-reference section of the application draft) containing the sequences of aptamers and proteins discussed in the specification. Each sequence is assigned a unique SEQ ID NO. (SEQ ID NO:1, 2, etc.).

### Fine-Tuning on Protein Families or Aptamer Classes

The training method further comprises a step (h) of fine-tuning the machine learning model on at least one selected molecular target protein family or on at least one aptamer class. This is a second-stage or specialized training step, wherein a model already trained on diverse aptamer-protein pairs is further optimized on a narrower, more specific domain.

Protein Family Fine-Tuning: After training on a broad dataset of aptamers against diverse protein targets, the model can be re-trained (with a lower learning rate and fewer epochs) on a dataset comprising aptamers against a specific protein family, such as:
a. Kinases: Protein targets with catalytic kinase domains, known to have similar ATP-binding pockets and substrate-binding grooves.
b. Transcription factors: Proteins with DNA-binding domains, often featuring conserved zinc fingers or helix-turn-helix motifs.
c. Antibodies: Immunoglobulin proteins with conserved constant regions and variable antigen-binding regions.
d. Enzymes: Proteins with specific active sites and substrate specificity.

Fine-tuning on a protein family allows the model to specialize to the structural and functional features of that family, potentially improving prediction accuracy for aptamers targeting family members.

Aptamer Class Fine-Tuning: Alternatively, or in addition, the model can be fine-tuned on a specific aptamer class, such as:
a. G-quadruplex-forming aptamers: Sequences known to form G-quadruplex structures, important for telomere-targeting and cancer therapeutics.
b. Hammerhead ribozyme aptamers: Catalytic aptamers incorporating ribozyme motifs.
c. Aptamers from a specific SELEX campaign: Sequences derived from a particular in vitro selection experiment, sharing common structural features or selection history.
d. DNA vs. RNA aptamers: Fine-tuning separate models for DNA and RNA, if they exhibit substantially different binding mechanisms.

Transfer Learning: Fine-tuning leverages transfer learning—the principle that knowledge acquired in one domain (broad aptamer-protein interactions) can accelerate learning in a related but more specialized domain (a specific protein family or aptamer class). Fine-tuning starts from a model already trained on general aptamer-protein data, requiring fewer training iterations and less data to reach high performance compared to training from scratch.

Practical Application: in one example, a research group specializing in aptamers to cancer-related protein kinases could fine-tune the general model on their internal dataset of kinase-binding aptamers, yielding a kinase-specialized model with high prediction accuracy for their domain of interest.

### Development and Architecture

In another embodiment of the disclosure, a method employs a symmetric architecture that processes both protein and aptamer sequences through specialized pretrained encoders (ESM2-650m lin2022language for proteins, nucleotide-transformer-v2-50m dalla2023nucleotide or RNA-FM chen2022interpretable for aptamers), followed by a fusion mechanism for combining embeddings into a shared aptamer-protein representation. It can comprise symmetric cross-attention modules that enable bidirectional information flow between protein and aptamer representations, capturing complex interaction patterns without providing any structural information. The fusion architecture comprises two cross-attention modules and two self-attention modules, the outputs of which are summed, pooled, and concatenated to arrive at a joint representation. This can be processed by a multi-layer perceptron to generate binding predictions.

The cross-attention module, adapted from xu2023protst, allows the system to capture both global (long-range) and local interactions across tokens. This demonstrates an improvement over conventional methods, where convolution mechanisms may be limited to capturing interactions within small communities of monomers. For generating an aptamer-attended protein representation, the disclosed method can pass the protein embedding as the queries Q and the aptamer embedding as key and value matrices K, V. The converse is done to generate a protein-attended aptamer representation. The disclosed method can also process embeddings via self-attention. These self-attended representations are added to the cross-attended representations to generate an attention-enhanced representation of both the protein and aptamer. The aforementioned cross-attended representations yield attention maps connecting amino acids to bases, providing a glance into which interactions most inform the model output.

### De Novo Generation

To generate aptamers de novo, the disclosure can adopt and extend two methods implemented via a Monte Carlo Tree Search (MCTS) where the present disclosure is used to compute rewards: sequence-space exploration via random sequence design lee2021 predicting and sequence-space exploration via diffusion-generated samples.

### De Novo Generation—MCTS (Monte Carlo Tree Search)

The first approach is a simple Monte Carlo Tree Search method \citep{lee2021 predicting} only requiring the use of the AptaBLE classifier. In addition to the below, the full algorithm is described in Algorithm 1.

### Initialization

The final aptamer is a sequence of fixed length \textit{N}, where initially the sequence is completely empty. At each iteration of the MCTS, a new token is either appended or prepended to the existing aptamer sequence based on the selection-expansion-rollout cycle followed by the algorithm.

### Selection

From the root node, the algorithm progresses down the MCTS tree based on the UCT (upper confidence bounds applied to trees) score, defined as the following:

$$U C {T}_{i , {s}_{i} , {n}_{i} , {N}_{i}} = \frac{{s}_{i}}{{n}_{i}} + C \sqrt{\frac{ln ( {N}_{i} )}{{n}_{i}}}$$

where i identifies the node, sᵢ is the accumulated exploitation reward for node i calculated during backpropagation, nᵢ is the number of visits to node i, Nᵢ is the number of visits to the parent node of node i, and C is a tunable exploration parameter set to 1/\sqrt{2} according to prior work \citep{lee2021 predicting}. Ultimately, a leaf node is reached following the optimal UCT path.

### Expansion

From the selected leaf node, a new child node is added. This reflects either the appending or prepending of a nucleotide base to the existing sequence represented by the currently selected node (i.e. _A, _C, _G, _T, A_, C_, G_, or T_, where _ denotes the current state of the node).

### Rollout

For each child node x^{child}^{ᵢ}, the remainder of the aptamer sequence is completed by randomly appending nucleotides from the model alphabet until the sequence reaches length N. This random sequence generation from the child node denotes one sample from the child node state. Ultimately, each sequence generated from this method reflects the child node's construction and is passed into AptaBLE alongside the target protein for score computation. This reflects an individual play-out.

### Backpropagation

For each child node x^{child}^{ᵢ}, the cumulative reward is updated as denoted by its UCT by populating the $s_i$ value using the AptaBLE scores corresponding to that play-out. This procedure of exploring random play-outs is repeated for the indicated number of iterations in the MCTS. Ultimately, the procedure repeats from the selection step, with the child node having the greatest UCT score being added to the ensuing selection path.

### Reward

The reward vector for a node consists of two scores, which equally contribute to the sᵢ value in the UCT computation. The first is the AptaBLE score against the target protein and the second is the AptaBLE score against an off-target protein subtracted from 1 to function as a ‘negative’ selection. These scores are equally weighted and combined, then serve as the sequence's exploitation reward within its UCT.

Another embodiment of the current disclosure is a method of applying Monte Carlo Tree Guidance to aptamer sequence design. This method requires the use of a classifier and an appropriate masked diffusion model to sample sequences from each explored state. In addition to the below, the full algorithm is described in Algorithm alg:mctg.

### Monte-Carlo Tree Guidance

The second approach is applying Monte Carlo Tree Guidance to aptamer sequence design. This method requires the use of the AptaBLE classifier and an appropriate masked diffusion model to sample sequences from each explored state. In addition to the below, the full algorithm is described in Algorithm 2.

Here, de novo generation requires the use of a masked diffusion model (MDM) for sampling sequences at the rollout stage.

To guide unconditionally-generated sequences generated by the MDM towards co-optimality across functionally-relevant aptamer properties, we leverage the Monte Carlo Tree Guidance (MCTG) algorithm introduced in \citet{tang2025peptune} for multiobjective guidance of discrete diffusion across several pre-trained regressor models.

### Initialization

A fully masked tokenized sequence is initialized with a length L. This becomes the root node of the MCTS tree with timestep t=0. Additionally, we construct a vector scoring function ƒ:V^{L}→R^{K} that returns a score vector given a clean input sequence x₀∈V^{L} and initialize an empty Pareto-optimal set P*={ }$. The following hyperparameters are defined: number of MCTS iterations Nᵢₜₑᵣ and number of children nodes N_{child}. The root node is initialized as a fully-masked sequence of length L.

### Selection

To determine the next unmasking step from a parent node with an already expanded set of child sequences xᵢ^{child}∈children(x), a selection score vector U(xᵢ^{child})=R(xᵢ^{child})/Nᵥᵢₛᵢₜ(xᵢ^{child}) is computed that divides the cumulative reward vector R(xᵢ^{child}) of a child node to the number of times it was visited Nᵥᵢₛᵢₜ(xᵢ^{child}). This encourages exploration of child nodes that have not been visited, in addition to high-reward nodes. Then, a selection is made from the Pareto-optimal set of child nodes based on the normalized selection score vector.

$${𝒫}_{s e lect}^{*} = \{ {x}_{i}^{child} \vert ∄ {x}_{j}^{child} ∈ children ( x ) s . t . U ( {x}_{j}^{child} ) ≻ U ( {x}_{i}^{child} )$$

If a non-leaf node is selected, the selection process is restarted with the selected node as the new parent until an expandable leaf node or a fully unmasked sequence is reached. In the case of a fully unmasked sequence, the selection is restarted from the root node.

### Expansion

From the leaf node xₜ at time t, N_{child} distinct sequences are sampled by applying Gumbel noise to the denoising distribution p_{0|t}^{θ}(x₀|xₜ)

$${x}_{0}^{i} ∼ log {p}_{0 \vert t}^{θ} ( {x}_{0} \vert {x}_{t} ) + {g}_{i}$$

where gᵢ~Gumbel(0,1). Then, for each child node

$${x}_{i}^{child} ,$$

k positions are randomly selected in xₜ to unmask according to

$${x}_{1}^{i} .$$

### Rollout

For each child node xᵢ^{child}, a self-planned ancestral sampling is used to obtain a fully unmasked sequence \tilde{x}ᵢ^{child}. This is in stark contrast to the aforementioned MCTS method, where sequences corresponding to each state are produced completely at random. Then, each of the clean sequences is fed into the vector score function ƒ(\tilde{x}ᵢ^{child}) to obtain a score vector. The reward vector r(xᵢ^{child})∈r^{k} of each child xᵢ^{child} is computed, where each element is the fraction of sequences in the Pareto-optimal set x*∈P* that xᵢ^{child} dominates.

$${r}_{k} ( {x}_{i}^{child} ) = \frac{1}{\vert {𝒫}^{*} \vert} {\sum }_{{x}^{*} ∈ {𝒫}^{*}} 1 [ {f}_{k} ( {x}_{i}^{child} ) \geq {f}_{k} ( {x}^{*} ) ]$$

For all N_{child} children sequences, xᵢ^{child} is added to P* if it is non-dominated by all sequences x*∈P* and remove all sequences in P* dominated by some xᵢ^{child}.

### Backpropagation

For each child node xᵢ^{child}, its reward vector r(xᵢ^{child}) is used to initialize the cumulative reward R(xᵢ^{child}) r(xᵢ^{child}) and the number of visits Nᵥᵢₛᵢₜ(xᵢ^{child}) is set to 1.

A backward tracing from xᵢ^{child} through its ancestors is performed up to the root node x₀^{root}, updating each node x along the path by accumulating the child rewards and incrementing the visit count:

$$W ( x ) ← W ( x ) + {\sum }_{i = 1}^{{N}_{c h i l d}} r ( {x}_{i}^{child} )$$

$${N}_{v isit} ( x ) ← N ( x ) + 1$$

These cumulative updates influence future selection, prioritizing unmasking paths that lead to higher-reward sequences for further expansion. After Nᵢₜₑᵣ iterations of these four steps, the set of aptamer sequences is returned while optimized across the set of properties x*∈P* and their corresponding score vectors ƒ(x*).

### Reward Vector

Similar to the MCTS method, the reward vector here reflects the AptaBLE scores for each aptamer against the target protein and an off-target protein serving as an \textit{in-silico} negative control (subtracted from 1). These scores are equally weighted and combined, forming the backpropagated reward.

### EXPERIMENTAL VALIDATION AND IMPLEMENTATION EXAMPLES

The following examples describe experiments conducted to demonstrate the reproducibility and performance of the disclosed target-conditioned aptamer discovery system. These examples illustrate representative embodiments and are not intended to limit the scope of the claims. Unless defined otherwise, parameters and conditions are standard for the art.

### Example 1: System Development and Architecture

In some embodiments, the system processes primary sequences of a protein molecular target and an aptamer candidate through specialized pretrained encoders to generate embeddings. A symmetric fusion module performs bidirectional cross-attention between protein and aptamer embeddings and further refines each with self-attention. The fused representation is pooled and reduced by a multilayer perceptron (MLP) to yield a binding-likelihood score in [0,1]. The architecture naturally accommodates variable-length aptamers without truncation artifacts.

Representative configuration (non-limiting): hidden size 512, 8 attention heads for both cross- and self-attention; ESM-2 embeddings reshaped from 1280 to 512 or 640 for compatibility; MLP mapping {1024 or 1280}→512→256→1 with ReLU and 1-D batch normalization between linear layers.

### Example 2: Training Datasets and Procedures

In some embodiments, training data comprise (i) experimentally characterized aptamer-protein pairs (positives defined, for example, as sub-micromolar Kd) and (ii) non-binding pairs, optionally including artificial negatives constructed by shuffling positive sequences to preserve length and nucleotide composition while disrupting secondary structure. To reduce information leakage, train/validation/test splits may be formed by sequence-homology clustering with a minimum identity and coverage threshold (e.g., 0.8/0.8).

A representative training environment used included a GPU-enabled compute instance (e.g., 80 GB A100 class GPU). In one embodiment, optimization employed AdamW with binary cross-entropy loss, initial learning rate about 1×10⁻⁵, and a MultiStepLR scheduler decaying by 0.1 every five epochs. Batch size was 16. Hyperparameters (learning rate, batch size, random seed) were swept over modest grids, and the best-performing checkpoint was selected by benchmark metrics.

### Example 3: De Novo Aptamer Generation Methods

### Monte Carlo Tree Search (MCTS)

In some embodiments, de novo ssDNA (or RNA) aptamers are generated by a classifier-guided MCTS in sequence space. Each node encodes a partial sequence; actions append or prepend one nucleotide. Selection uses an upper-confidence-bound policy over cumulative reward; expansion adds a single base; rollout stochastically completes to a fixed length; backpropagation updates node statistics. A reward vector may include (a) AptaBLE score for the intended protein target and (b) 1 minus the AptaBLE score for an off-target, thereby implementing negative selection.

In some embodiments, the algorithm for MCTS is one illustrated in FIG. 6.

3.2 Monte Carlo Tree Guidance (MCTG) with Masked Diffusion

In some embodiments, a masked discrete diffusion model samples sequence candidates at each rollout. Child states are created by unmasking token positions under a denoiser; samples are scored by a vector scoring function, and Pareto-optimal sequences are retained (e.g., target affinity vs. off-target non-binding). Rewards propagate up the tree to favor unmasking paths that yield higher multiobjective quality.

In some embodiments, the algorithm for MCTG is the one illustrated in FIG. 8.

In some embodiments of MCTG unless stated otherwise, the denoiser operates over the DNA alphabet {A,C,G,T,N}; if an RNA denoiser is used, we map U→T after denoising before scoring. MCTG may use a linear masking schedule k(t)=┌α·(t+1)┐ with α∈[0.05,0.25] (typ.), until all L positions are unmasked or a max depth T_max is reached. Selection among Pareto-optimal children uses a normalized weighted scalarization Σ_j w_j·U_j(u) (e.g., w=[0.5,0.5] for target/off-target) as a deterministic tie-break. MCTG Typical settings: N_iter=5e3-5e4, N_child=4-16, T_max=L/α.

### Example 4: Benchmarking and Ablation Studies (Computational)

In some embodiments, AptaBLE is benchmarked against sequence-based and structure-based baselines across DNA and RNA panels withheld by target and sequence identity. Representative results observed include improved ROC-AUC and Matthews correlation coefficient relative to sequence-only baselines that employ dot-product fusion, and improved precision relative to structure-based pipelines at large-scale screening throughput. Ablations replacing bidirectional cross-attention with (i) self-only attention, (ii) unidirectional attention, or (iii) simple concatenation/dot-product fusion produced measurable performance drops, supporting the contribution of symmetric cross-attention to generalization.

### Example 5: Wet-Lab Validation—CD117 (c-KIT) Case Study

### 5.1 SELEX Procedure (Representative)

In some embodiments, ssDNA aptamers were evolved against human CD117 using a randomized N26 library with negative selections (e.g., human serum albumin and 6×His peptide). PCR amplification and strand separation followed standard protocols. Libraries were thermally folded prior to selection in a defined selection buffer. After nine rounds under increasing stringency, the R9 pool was sequenced.

### Library Analysis and Candidate Selection

Sequence reads were clustered by edit distance (e.g., FastAptamer), yielding a set of clusters and a large remainder of unclustered sequences. The entire R9 library was scored by the system. A thresholding strategy (e.g., model score 0.4 and minimum copy number 10) prioritized unclustered candidates for synthesis alongside representative clustered sequences. Secondary-structure predictions and similarity to known binders informed the final pick list.

### Binding Assays

Binding affinities were measured by biolayer interferometry (BLI) at ~30° C. under standard buffer conditions. Aptamers were tested at serial concentrations with typical association and dissociation phases (e.g., 900 s each). Dissociation constants (Kd) were fit under a 1:1 binding model where appropriate. In some embodiments, aptamers are provided with sequence identifiers for ST.26 cross-reference.

### Aptamer (SEQ ID NO: _) Clustered Kd (M)

a. ABWUC1 (SEQ ID NO: 1) No 1.99×10⁻⁸
b. ABWUC2 (SEQ ID NO: 2) No 3.26×10⁻⁸
c. ABWUC3 (SEQ ID NO: 3) No ND*
d. ABWUC4 (SEQ ID NO: 4) No 6.02×10⁻⁷
e. ABW02 (SEQ ID NO: 5) Yes 2.10×10⁻⁸
f. ABW03 (SEQ ID NO: 6) Yes 6.18×10⁻⁷
g. ABW04 (SEQ ID NO: 7) Yes 1.10×10⁻⁶
h. ABW05 (SEQ ID NO: 8) Yes 3.29×10⁻⁷
i. ABW06 (SEQ ID NO: 9) Yes 1.53×10⁻⁵
j. ABW07 (SEQ ID NO: 10) Yes 1.22×10⁻⁹
k. *ND=not determined or no reliable fit under assay conditions.

In this study, all four of the system-prioritized unclustered candidates bound CD117 by BLI, and two (ABWUC1, ABWUC2) ranked among the top performers, demonstrating that sequence-abundance-centric clustering may omit functional binders that the system can recover.

### Interpretation

These experiments support that the disclosed system can (i) screen and rank SELEX libraries, (ii) uncover high-affinity candidates overlooked by copy-number heuristics, and (iii) guide wet-lab validation efficiently. In some embodiments, modest target-specific fine-tuning of the prediction head further aligns scores with measured Kd rank ordering.

These binding affinities and Kd values are provided by way of example only and are not intended to limit the scope of the disclosure. A person of ordinary skill in the art will recognize that aptamers with higher or lower measured affinity, different kinetic profiles, or context-dependent Kd values (e.g., assay-dependent variation) can still fall within the scope of the disclosed methods and systems, provided they exhibit specific binding to the target under relevant conditions.

### Example 6: Workflows and Utilities

In some embodiments, the system reduces SELEX rounds by enabling earlier prioritization of selective binders and by down-ranking sequences with high off-target risk using in-silico negative selection. Additional contemplated utilities include:
a. Affinity maturation via in-silico mutagenesis with model-guided selection.
b. Minimum-motif discovery by truncation/k-mer search maintaining predicted activity.

Generative design (MCTS, MCTG) of target-specific aptamers with Pareto trade-offs (affinity, specificity, length, manufacturability).

These workflows are compatible with purely computational discovery or hybridized with experimental validation.

### Example 7: Representative Aptamer Sequences and Binding Characterization

Identification of Aptamers cd25-73 (SEQ ID NO:17) and cd25-77 (SEQ ID NO:18)

In certain embodiments, the disclosure provides aptamer sequences identified for selective recognition of the CD25 antigen. Aptamer cd25-73, corresponding to SEQ ID NO:17, was discovered using the Monte Carlo Tree Guidance (MCTG) method described herein. Aptamer cd25-77, corresponding to SEQ ID NO:18, was discovered using the Monte Carlo Tree Search (MCTS) method described herein. These sequences were subsequently evaluated for binding affinity and selectivity using the experimental protocol below.

### Experimental Protocol for Aptamer Binding to CD25-Expressing Cells

To assess the specificity and affinity of FAM-labeled aptamers corresponding to SEQ ID NO:17 and SEQ ID NO:18 toward CD25-positive cells, a comparative binding assay was performed using Ramos (CD25+) cells and HEK293T (CD25−) cells as a negative control.

### Aptamer Preparation and Folding

Aptamers were dissolved in nuclease-free water at a stock concentration of 1 mM. A 50 μM working solution was prepared in folding buffer containing 20 mM HEPES (pH 7.4), 150 mM NaCl, and 5 mM MgCl₂. Aptamers were thermally folded by heating at 95° C. for 5 minutes, cooling at 4° C. for 5 minutes, incubating at 25° C. for 5 minutes, and holding again at 4° C. for 5 minutes. Folded aptamers were serially diluted 4-fold in 1× selection buffer to yield concentrations ranging from 50 μM to 0.781 μM.

### Cell Preparation

Ramos cells were harvested, centrifuged at 400×g for 10 minutes, and washed in 1× selection buffer containing 20 mM HEPES (pH 7.4), 150 mM NaCl, 5 mM MgCl₂, and 10% FBS. Cell concentration and viability were assessed by Trypan Blue staining. HEK293T cells were cultured and processed in parallel under identical conditions.

### Binding Assay Setup

A 96-well plate was prepared with 10,000 cells per well in a final volume of 100 μL. Volumes of selection buffer and cell suspension were adjusted accordingly to yield 90 μL prior to aptamer addition. Ten microliters of each folded aptamer dilution were added to designated wells. Six replicates were performed for each concentration. Wells containing cells without aptamer served as background fluorescence controls. Plates were gently mixed and incubated at 37° C. with 5% CO₂ for 40 minutes.

### Washing Procedure

Plates were centrifuged at 450×g for 10 minutes, the supernatant was removed, and cells were resuspended in 200 μL of 1× selection buffer.

### Flow Cytometry Acquisition

Flow cytometry was performed using an Attune cytometer equipped with a 488 nm laser and BL1 detector for FAM signal. Standard gating strategies (FSC-A vs. SSC-A and FSC-H vs. FSC-A) were applied to identify viable single cells. A minimum of 5,000-10,000 events per sample were collected. Binding was quantified by comparing median fluorescence intensity (MFI) values between Ramos and HEK293T cells.

### Kd Determination

Equilibrium dissociation constants (Kd) were determined by fitting MFI versus aptamer concentration to a one-site specific binding model using nonlinear regression (GraphPad Prism). Data were reported as best-fit values with associated standard errors.

### Results

Binding to CD25-Positive Ramos Cells
Aptamer cd25-73 (SEQ ID NO:17): 0.7658±0.2417 μM
Aptamer cd25-77 (SEQ ID NO:18): 0.03080±0.01224 μM

Aptamer cd25-77, identified using the MCTS method, demonstrated the highest affinity, with a sub-100 nM dissociation constant.

### Binding to CD25-Negative HEK293T Cells

Aptamer cd25-73 (SEQ ID NO:17): 48.11±224.9 μM (low-confidence fit)

a. Aptamer Cd25-77 (SEQ ID NO:18): Poor Fit (No Reliable Binding Detected)

The negligible binding observed for both aptamers with CD25-negative HEK293T cells demonstrates selective recognition of the CD25 target, with cd25-77 exhibiting especially high specificity. This result illustrates one embodiment of the disclosed system's ability to identify high-affinity aptamers, but the invention is not limited to aptamers exhibiting sub-100 nM Kd. Any aptamer identified or optimized using the disclosed computational methods that shows specific, target-selective binding is contemplated.

### Example 8: Predicted Aptamer Sequences Identified by Computational Design Methods

In certain embodiments, the disclosure provides aptamer sequences generated through computational design workflows, including Monte Carlo Tree Search (MCTS) and Monte Carlo Tree Guidance (MCTG) methods described herein. These sequences are provided as SEQ ID NOs. 11-13. Each sequence was identified by applying the disclosed scoring and generative framework to a designated protein target. Although these sequences have not yet undergone experimental wet-lab validation, they exhibit computationally predicted binding properties and are expected, based on model scoring, to interact with their respective protein targets.

SEQ ID NO:11, referred to as ABWT08-C, was identified using the MCTS-based sequence exploration method. This sequence was computationally optimized against the receptor binding domain (RBD) of the SARS-CoV-2 spike protein. The disclosed workflow assigns a predicted interaction score based on cross-attended aptamer-protein embeddings, suggesting that SEQ ID NO:11 may possess structural and sequence features conducive to RBD binding.

SEQ ID NO:12, referred to as ABWT09-C, was likewise identified through MCTS-guided de novo design. This sequence was generated and selected for predicted affinity toward basic fibroblast growth factor (bFGF). Scoring derived from the disclosed model indicates that SEQ ID NO:12 contains nucleotide motifs and interaction patterns computationally associated with bFGF recognition.

SEQ ID NO:13, referred to as ABWT11-C, was identified using the MCTG framework, which employs guided diffusion sampling combined with multiobjective scoring. SEQ ID NO:13 was designed toward the human CD4 (hCD4) protein target. The MCTG process applies Pareto optimization across multiple computational objectives, resulting in predicted high-quality binding characteristics for this sequence.

The computational approaches used to identify these sequences evaluate long-range token interactions, predicted structural compatibility, electrostatic complementarity, and cross-attention alignment patterns between aptamer and protein embeddings. Accordingly, SEQ ID NOs. 11-13 exhibit predicted binding capacity to their respective targets (SARS-CoV-2 RBD, bFGF, and hCD4). A person of ordinary skill in the art would be able to experimentally assess and confirm these predicted interactions using routine binding assays, such as flow cytometry, filter binding, fluorescence anisotropy, SPR, or ELISA-style plate binding formats.

### Example 9

The aptamer sequences hCD4-54 (SEQ ID NO:14), hCD4-72 (SEQ ID NO:15), and hCD4-121 (SEQ ID NO:16) were generated using a Monte Carlo Tree Search (MCTS) system-based aptamer design algorithm configured with human CD4 (hCD4) as the target protein. These sequences represent model-guided, target-conditioned candidates produced through iterative exploration and optimization steps within the MCTS framework. Each of the three aptamers was subsequently validated through the experimental procedures described herein, confirming their binding activity toward hCD4.

The CD4-binding DNA aptamer sequences selected from hCD4-54 (also referred to as Apt54; SEQ ID NO:14), hCD4-72 (also referred to as Apt72; SEQ ID NO:15), and hCD4-121 (also referred to as Apt121; SEQ ID NO:16) were designed using a MCTS system. Each of these aptamers is a 40-nucleotide single-stranded DNA oligonucleotide whose sequence is set forth in the accompanying sequence listing under the indicated Sequence ID Numbers. The aptamers were designed using a sequence first aptamer protein interaction platform (e.g., an AptaBLE-MCTS model) that accepts as input the amino acid sequence of recombinant human CD4 (rCD4) and iteratively explores a 40-mer randomized nucleic acid search space (N40 library) using Monte Carlo tree search (MCTS) guided by a neural network scoring function trained on aptamer protein binding datasets. Candidate sequences predicted to bind hCD4 with high affinity and to exhibit reduced nonspecific interactions were identified in silico by (i) ranking predicted binding scores against hCD4, (ii) applying counter-selection against human serum albumin, and (iii) filtering for favorable biophysical properties including GC content and predicted minimum free energy. For structural vetting, the secondary structures of all candidates were predicted (e.g., using mFold) and hierarchically clustered, and tertiary docking of the aptamer-hCD4 complexes was evaluated (e.g., using Chai-1), thereby identifying hCD4-54, hCD4-72, and hCD4-121 as structurally diverse aptamers predicted to bind a similar epitope on the hCD4 surface as a benchmark CD4 aptamer.

To prepare the aptamers for nanoparticle display, each of hCD4-54, hCD4-72, and hCD4-121 may be synthesized as a 5′-thiol-modified DNA oligonucleotide (e.g., via solid-phase synthesis with a terminal thiol modifier) and purified by HPLC. Prior to conjugation, the aptamers are folded in an aptamer folding buffer comprising, for example, 50 mM HEPES, 5 mM MgCl₂, and 150 mM NaCl at pH 7.4 by heating to about 95° C., rapidly cooling to about 4° C., and then slowly warming to about 25° C. If present, disulfide-protected thiols can be reduced by incubation with a reducing agent such as tris(2-carboxyethyl)phosphine (TCEP) at a molar ratio of about 1:10 (aptamer:TCEP), followed by buffer exchange into 1×PBS (pH 7.5) and centrifugal filtration to remove excess reagent. Lipid nanoparticles (LNPs) suitable for aptamer display can be formulated using an ionizable lipid such as SM102, a helper phospholipid (e.g., DSPC), cholesterol, and a PEGylated lipid (e.g., PEG2000-DMG and/or DSPE-PEG2000-maleimide) in molar ratios of about 50:10:38.5:1.2:0.3, respectively, and used to encapsulate an mRNA cargo (e.g., encoding eGFP) at an N/P ratio of about 10. LNPs can be prepared, for example, by microfluidic mixing of an ethanolic lipid phase with an aqueous mRNA phase at a flow ratio of about 1:3, followed by dialysis into 1×PBS (pH 7.5) and concentration by ultrafiltration.

Conjugation of hCD4-54, hCD4-72, or hCD4-121 to the LNP surface may be performed via thiol-maleimide chemistry under conditions that maintain particle integrity. In one non-limiting configuration, the maleimide-bearing LNPs are mixed with reduced, folded aptamer at a stoichiometric ratio selected to yield a theoretical aptamer loading of about 100 aptamers per LNP (e.g., a 100:1 aptamer-to-particle ratio), and the reaction is incubated at room temperature until completion. Aptamer density on the LNP surface may be quantified using a complementary molecular beacon assay in which a fluorescently labeled DNA beacon specific to a unique loop region of each aptamer is hybridized to the aptamer-LNPs and fluorescence is compared to a standard curve generated with free aptamer. Dynamic light scattering (DLS) can be used to verify that conjugation maintains an average LNP diameter of about 90-110 nm and a polydispersity index (PDI) below about 0.2, with only minimal shifts in zeta potential relative to non-targeted LNPs.

The functional targeting activity of hCD4-54, hCD4-72, and hCD4-121 can be demonstrated using CD4⁺ and CD4⁻ T-cell lines. In one exemplary protocol, SM102 LNPs encapsulating eGFP mRNA are conjugated to each aptamer at a 100:1 aptamer-to-LNP ratio and added to CD4⁺ SUP-T1 cells and CD4⁻ HSB-2 cells at a dose of about 500 ng mRNA per 100,000 cells. Cells are incubated for approximately 24 hours at 37° C. in a humidified 5% CO₂ incubator, washed to remove non-internalized particles, and analyzed for eGFP fluorescence (e.g., by plate reader and/or flow cytometry). In representative implementations, hCD4-54-, hCD4-72-, and hCD4-121-conjugated LNPs produce significantly elevated eGFP expression in CD4⁺ SUP-T1 cells relative to CD4⁻ HSB-2 cells, with statistical significance assessed, for example, by two-way ANOVA followed by Tukey's multiple-comparisons test (e.g., ****p<0.0001). At the same time, cell viability measured by a luminescent ATP assay remains above about 80% across relevant dose ranges, indicating that aptamer display does not introduce unacceptable cytotoxicity under these conditions. These data show that hCD4-54, hCD4-72, and hCD4-121 function as CD4-selective targeting ligands when displayed on LNPs and enable sequence-specific delivery of mRNA cargo to CD4⁺ T-cell populations.

These predicted aptamer sequences are included as part of the sequence landscape generated by the disclosed computational methods. Their presence enables support for further embodiments, including experimental validation, optimization, truncation variants, chemical modification variants, or future claims directed to aptamers that bind the corresponding protein targets.

Sequence Listings: Aptamer sequences referenced herein should be provided in the XML sequence listing and cross-referenced above as SEQ ID NOs.

In some embodiments, the following training details were used: Python 3.9, PyTorch 2.3.x, GPU memory 48 GB, batch size 16, AdamW optimizer, initial learning rate ~1×10⁻⁵ with MultiStepLR (decay 0.1 every five epochs). Equivalent libraries, frameworks, or hardware may be substituted without departing from the scope of the invention.

As used herein, the term “AptaBLE” refers to the target-conditioned aptamer-protein binding prediction system, including its underlying neural network architecture, generative components, and associated computational pipelines disclosed in this application. The term “AptaBLE” is used for convenience only and is not intended to be limiting. The disclosure may refer interchangeably to “AptaBLE,” “the disclosed model,” “the disclosed system,” “the aptamer-protein binding model,” or “the target-conditioned aptamer discovery system,” all of which shall be understood to refer to the same apparatus, methods, and implementations described herein.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12, 15, 20, or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims.

What is claimed is:

1. A computer-implemented method for predicting aptamer-protein target binding, comprising the following steps:
(a) obtaining a vector representation of an aptamer sequence, further comprising a step of tokenizing the aptamer sequence into discrete aptamer units and a step of mapping each aptamer unit to an aptamer numerical vector,
(b) obtaining a vector representation of a molecular target protein sequence, further comprising a step of tokenizing the molecular target protein sequence into discrete protein units and a step of mapping each protein unit to a protein numerical vector,
(c) generating an aptamer embedding using an aptamer encoder based on the vector representation of the aptamer sequence of step (a),
(d) generating a molecular target protein embedding using a protein encoder based on the vector representation of the protein sequence of step (b);
(e) processing the aptamer embedding of step (c) and the molecular target protein embedding of step (d) using a neural network comprising a self-attention network with a plurality of self-attention network layers and a cross-attention network with a plurality of cross-attention network layers; and
(f) generating a prediction score indicating a likelihood of binding between the aptamer sequence and the protein molecular target sequence based on outputs of the cross-attention network of step (e).

2. The computer-implemented method of claim 1, wherein step (c) further comprises using an aptamer transformer model, and step (d) further comprised using a protein transformer model.

3. The computer-implemented method of claim 1, wherein step (e) further comprises the following steps:
analyzing internal relationships within the aptamer embedding and within the molecular target protein embedding using the self-attention network; and
modeling interactions between the aptamer embedding and the molecular target protein embedding using the cross-attention network.

4. The computer-implemented method of claim 1, wherein step (f) further comprises a step of processing the outputs of the cross-attention network of step (e) through a multi-layer perceptron to produce a value between 0 and 1.

5. The computer-implemented method of claim 1, further comprising:
(g) identifying a specific residue on the molecular target protein sequence with a highest contribution to the predicted binding interaction, and identifying a specific nucleotide on the aptamer sequence with a highest contribution to the predicted binding interaction.

6. A target-conditioned candidate aptamer prediction apparatus, comprising:
a processor unit;
a memory unit storing a machine learning model, wherein the machine learning model, in turn, comprising:
a vectorization component configured to generate a vector representation of an aptamer sequence and a vector representation of a molecular target protein sequence,
an aptamer encoder configured to generate an aptamer embedding from the vector representation of the aptamer sequence,
a molecular protein target encoder configured to generate a molecular target protein embedding from the vector representation of the molecular target protein sequence,
a self-attention network configured to individually process each of the aptamer embedding and the molecular target protein embedding to model internal molecular relationships therein; and
a cross-attention network configured to process relationships between the aptamer embedding and the molecular target protein embedding by relating protein molecular target query token embeddings to aptamer key-value embeddings, the cross-attention network is further configured to generate combined representations of the processed relationships.

7. The apparatus of claim 6, wherein the machine learning model further comprises a nucleic acid encoder for DNA aptamer embedding.

8. The apparatus of claim 6, further comprising a training unit configured to optimize parameters of the machine learning model to fit experimental binding data quantified via a convex, differentiable loss function.

9. The apparatus of claim 6, wherein the memory unit is configured to store representations of critical motifs for protein target-binding, wherein representations of critical motifs for protein target-binding are adapted to be referenced and arranged when prompted using a novel protein sequence.

10. The apparatus of claim 6, further comprising a multi-layer perceptron configured to generate a binding prediction score.

11. The apparatus of claim 6, wherein the processor unit is configured to iteratively modify the aptamer sequences to optimize predicted binding scores.

12. The apparatus of claim 6, further comprising an input/output (I/O) module configured to receive sequence inputs and provide visualization of predicted binding interfaces.

13. A method of training a machine learning model for predicting an aptamer-protein target binding, comprising the following steps:
(a) curating a training dataset and a benchmark dataset comprising known and proprietary aptamer-protein target pairs and corresponding binding data, in addition to known natural oligonucleotide-target complexes,
(b) generating a vector representation of at least one aptamer-protein target pair,
(c) generating an aptamer-protein embedding for the at least one aptamer-protein target pair,
(d) processing the aptamer-protein embedding through a self-attention network and a cross-attention network,
(e) comparing at least one predicted binding score with experimental binding data, and
(f) updating at least one parameter of the machine learning model using gradient-based optimization, using the comparison of step (e), thereby minimizing aptamer-protein binding prediction error.

14. The method of claim 13, wherein the training dataset comprises at least one negative example of a non-binding aptamer-protein target pair.

15. The method of claim 13, wherein step (f) further comprises a step of using backpropagation through the self-attention network, the cross-attention network, and an encoder used for generating the aptamer-protein embedding of step (c).

16. The method of claim 13, further comprising a step (g) of validating machine learning model performance using a held-out test set of aptamer-protein target pairs.

17. The method of claim 13, wherein the training dataset comprises binding affinity measurements obtained through experimental methods.

18. The method of claim 13, wherein the training dataset comprises experimentally-measured binding affinities for aptamers corresponding to SEQ ID NO:1, SEQ ID NO:2, SEQ ID NO:4, SEQ ID NO:17, or SEQ ID NO:18, wherein the experimentally-measured binding affinities serve as labeled training data for optimizing the machine learning model.

19. The method of claim 13, further comprising a step (h) of fine-tuning the machine learning model on at least one selected molecular target protein family or on at least one aptamer class.


SEQUENCE LISTING (18 sequences)

SEQ ID NO: 1 - DNA, 40 bp, unidentified
  source 1..40: mol_type = other DNA
ccgtgctagctaagttattgaggggagcaggatcaacacc

SEQ ID NO: 2 - DNA, 40 bp, unidentified
  source 1..40: mol_type = other DNA
tgaaaaacagcgaagtgagtccgcatatcgctgctgcgaa

SEQ ID NO: 3 - DNA, 50 bp, unidentified
  source 1..50: mol_type = other DNA
gcactgctgacgagtcccaaactaggacgaacgaaatgtgcgtcaagtgc

SEQ ID NO: 4 - DNA, 40 bp, unidentified
  source 1..40: mol_type = other DNA
cccgcacctagcggaatggtggcagctatcagaagtaatt

SEQ ID NO: 5 - DNA, 40 bp, unidentified
  source 1..40: mol_type = other DNA
cggagtcggtgttgtaaagaaggatgaaccgcatggtacc

SEQ ID NO: 6 - DNA, 40 bp, unidentified
  source 1..40: mol_type = other DNA
tatgctactactccgtacgcattgtctaagtgactgcaat

SEQ ID NO: 7 - DNA, 69 bp, unidentified
  source 1..69: mol_type = other DNA
atccagagtgacgcagcaccgcccatgtcgaataggtgccacgtagggacttggacacggtggcttagt

SEQ ID NO: 8 - DNA, 76 bp, unidentified
  source 1..76: mol_type = other DNA
atccagagtgacgcagcaccatgctaagtaccctggagtgctgtgccatctgtgtttatggacacggtggcttagt

SEQ ID NO: 9 - DNA, 77 bp, unidentified
  source 1..77: mol_type = other DNA
atccagagtgacgcagcacgagccttgtgtcgttcggagtgctggtccacgactactattggacacggtggcttagt

SEQ ID NO: 10 - DNA, 76 bp, unidentified
  source 1..76: mol_type = other DNA
atccagagtgacgcagcacagtccatgtcgaataggtgcgctgtcccatggatttcgttggacacggtggcttagt

SEQ ID NO: 11 - DNA, 77 bp, unidentified
  source 1..77: mol_type = other DNA
atccagagtgacgcagcacgagccttgtgtcgttcggagtgctgtgccagactggaatatggacacggtggcttagt

SEQ ID NO: 12 - DNA, 75 bp, unidentified
  source 1..75: mol_type = other DNA
atccagagtgacgcagcaccgtccatgtcgaataggctgctggtccactgacatttttggacacggtggcttagt

SEQ ID NO: 13 - DNA, 76 bp, unidentified
  source 1..76: mol_type = other DNA
atccagagtgacgcagcaccgcccatgtcgaatagatccagagtgacgcagcaccgtccatgtcgaataggtgtgc

SEQ ID NO: 14 - DNA, 76 bp, unidentified
  source 1..76: mol_type = other DNA
atccagagtgacgcagcaccgcccatgtcgaatagtatccagagtgacgcagcaccgcccatgtcgaataggtgtg

SEQ ID NO: 15 - DNA, 76 bp, unidentified
  source 1..76: mol_type = other DNA
atccagagtgacgcagcaccgtccatgtcgaatagccagagtgacgcagcaccgcccatgtcgaataggtgtgctg

SEQ ID NO: 16 - DNA, 76 bp, unidentified
  source 1..76: mol_type = other DNA
atccagagtgacgcagcacgagccttgtgtcgttctatccagagtgacgcagcaccgcccatgtcgaataggtgcc

SEQ ID NO: 17 - DNA, 40 bp, unidentified
  source 1..40: mol_type = other DNA
acccaggcacgacgaccacctaacacgtggtagcggtgcg

SEQ ID NO: 18 - DNA, 40 bp, unidentified
  source 1..40: mol_type = other DNA
atgaatgggtttcgtaggggagggacccactacactccaa